SEQUENCE LISTING (6 sequences)

SEQ ID NO: 1 - protein, 356 aa, Homo sapiens
MTKSYSESGLMGEPQPQGPPSWTDECLSSQDEEHEADKKEDDLETMNAEEDSLRNGGEEEDEDEDLEEEEEEEEEDDDQKPKRRGPKKKKMTKARLERFKLRRMKANARERNRMHGLNAALDNLRKVVPCYSKTQKLSKIETLRLAKNYIWALSEILRSGKSPDLVSFVQTLCKGLSQPTTNLVAGCLQLNPRTFLPEQNQDMPPHLPTASASFPVHPYSYQSPGLPSPPYGTMDSSHVFHVKPPPHAYSAALEPFFESPLTDCTSPSFDGPLSPPLSINGNFSFKHEPSAEFEKNYAFTMHYPAATLAGAQSHGSIFSGTAAPRCEIPIDNIMSFDSHSHHERVMSAQLNAIFHD

SEQ ID NO: 2 - protein, 272 aa, Homo sapiens
MFVKSETLELKEEEDVLVLLGSASPALAALTPLSSSADEEEEEEPGASGGARRQRGAEAGQGARGGVAAGAEGCRPARLLGLVHDCKRRPSRARAVSRGAKTAETVQRIKKTRRLKANNRERNRMHNLNAALDALREVLPTFPEDAKLTKIETLRFAHNYIWALTETLRLADHCGGGGGGLPGALFSEAVLLSPGGASAALSSSGDSPSPASTWSCTNSPAPSSSVSSNSTSPYSCTLSPASPAGSDMDYWQPPPPDKHRYAPHLPIARDCI

SEQ ID NO: 3 - protein, 236 aa, Homo sapiens
MESSAKMESGGAGQQPQPQPQQPFLPPAACFFATAAAAAAAAAAAAAQSAQQQQQQQQQQQQAPQLRPAADGQPSGGGHKSAPKQVKRQRSSSPELMRCKRRLNFSGFGYSLPQQQPAAVARRNERERNRVKLVNLGFATLREHVPNGAANKKMSKVETLRSAVEYIRALQQLLDEHDAVSAAFQAGVLSPTISPNYSNDLNSMAGSPVSSYSSDEGSYDPLSPEEQELLDFTNWF

SEQ ID NO: 4 - protein, 464 aa, Homo sapiens
MRLSKTLVDMDMADYSAALDPAYTTLEFENVQVLTMGNDTSPSEGTNLNAPNSLGVSALCAICGDRATGKHYGASSCDGCKGFFRRSVRKNHMYSCRFSRQCVVDKDKRNQCRYCRLKKCFRAGMKKEAVQNERDRISTRRSSYEDSSLPSINALLQAEVLSRQITSPVSGINGDIRAKKIASIADVCESMKEQLLVLVEWAKYIPAFCELPLDDQVALLRAHAGEHLLLGATKRSMVFKDVLLLGNDYIVPRHCPELAEMSRVSIRILDELVLPFQELQIDDNEYAYLKAIIFFDPDAKGLSDPGKIKRLRSQVQVSLEDYINDRQYDSRGRFGELLLLLPTLQSITWQMIEQIQFIKLFGMAKIDNLLQEMLLGGSPSDAPHAHHPLHPHLMQEHMGTNVIVANTMPTHLSNGQMSTPETPQPSPPGGSGSEPYKLLPGAVATIVKPLSAIPQPTITKQEVI

SEQ ID NO: 5 - protein, 457 aa, Homo sapiens
MLGAVKMEGHEPSDWSSYYAEPEGYSSVSNMNAGLGMNGMNTYMSMSAAAMGSGSGNMSAGSMNMSSYVGAGMSPSLAGMSPGAGAMAGMGGSAGAAGVAGMGPHLSPSLSPLGGQAAGAMGGLAPYANMNSMSPMYGQAGLSRARDPKTYRRSYTHAKPPYSYISLITMAIQQSPNKMLTLSEIYQWIMDLFPFYRQNQQRWQNSIRHSLSFNDCFLKVPRSPDKPGKGSFWTLHPDSGNMFENGCYLRRQKRFKCEKQLALKEAAGAAGSGKKAAAGAQASQAQLGEAAGPASETPAGTESPHSSASPCQEHKRGGLGELKGTPAAALSPPEPAPSPGQQQQAAAHLLGPPHHPGLPPEAHLKPEHHYAFNHPFSINNLMSSEQQHHHSHHHHQPHKMDLKAYEQVMHYPGYGSPMPGSLAMGPVTNKTGLDASPLAADTSYYQGVYSRPIMNSS

SEQ ID NO: 6 - protein, 443 aa, Homo sapiens
MYQSLAMAANHGPPPGAYEAGGPGAFMHGAGAASSPVYVPTPRVPSSVLGLSYLQGGGAGSASGGASGGSSGGAASGAGPGTQQGSPGWSQAGADGAAYTPPPVSPRFSFPGTTGSLAAAAAAAAAREAAAYSSGGGAAGAGLAGREQYGRAGFAGSYSSPYPAYMADVGASWAAAAAASAGPFDSPVLHSLPGRANPAARHPNLVDMFDDFSEGRECVNCGAMSTPLWRRDGTGHYLCNACGLYHKMNGINRPLIKPQRRLSASRRVGLSCANCQTTTTTLWRRNAEGEPVCNACGLYMKLHGVPRPLAMRKEGIQTRKRKPKNLNKSKTPAAPSGSESLPPASGASSNSSNATTSSSEEMRPIKTEPGLSSHYGHSSSVSQTFSVSAMSGHGPSIHPVLSALKLSPQGYASPVSQSPQTSSKQDSWNSLVLADSHGDIITA

(19) United States
(12) Patent Application Publication
(10) Pub. No.: US 2022/0152224 A1
(43) Pub. Date: May 19, 2022

(54) METHODS AND MATERIALS FOR TREATING CANCER

(71) Applicant: The Penn State Research Foundation, University Park, PA (US)

(72) Inventors: Gong CHEN, State College, PA (US); Xin WANG, State College, PA (US); Zifei PEI, State College, PA (US)

(21) Appl. No.: 17/439,733

(22) PCT Filed: Mar. 26, 2020
(86) PCT No.: PCT/US2020/024976

§ 371 Date: Sep. 15, 2021

Related U.S. Application Data
(60) Provisional application No. 62/823,702, filed on Mar. 26, 2019.

(51) Int. Cl. A61K 48/00; A61P 35/00

(52) U.S. Cl. CPC A61K 48/005; A61P 35/00

(57) ABSTRACT

This document relates to methods and materials for treating a mammal having cancer. For example, methods and materials for converting one or more cancer cells present in a mammal with cancer into non-cancerous cells are provided.

### CROSS-REFERENCE TO RELATED APPLICATIONS

This application claims the benefit of U.S. Patent Application Ser. No. 62/823,702, filed on Mar. 26, 2019. The disclosure of the prior application is considered part of (and is incorporated by reference in) the disclosure of this application.

### TECHNICAL FIELD

This document relates to methods and materials for treating a mammal having cancer. For example, this document provides methods and materials for converting one or more cancer cells present in a mammal with cancer into non-cancerous cells.

### BACKGROUND INFORMATION

Cancer is a major public health issue. In the United States alone over 1.7 million new cases were diagnosed in 2019 (National Cancer Institute, “Cancer Stat Facts: Cancer of Any Site,” at “https” colon “seer” dot “cancer” dot “gov/statfacts/html/all.html”.

Glioblastoma (GBM), a type of tumor that arises from unchecked proliferation of glial cells, accounts for half of the malignant brain tumor cases and has a 3.6 percent five-year relative survival rate (Ostrom et al., Neuro Oncol.; 17:ivl-iv62 (2015); and Porter et al., Neuroepidemiology.; 36(4):230-239 (2011)). Traditional therapies, such as chemotherapeutics, radiation therapy, and surgery, often fail in GBM because of active cell proliferation, invasive nature, and genomic and epigenetic heterogeneity (Brennan et al., Cell; 155(2):462 (2013); and McLendon et al., Nature; 455(7216):1061-1068 (2008)).

Worldwide, liver cancer (or hepatocellular carcinoma (HCC)) ranks third in cancer related deaths and sixth in incidence (El-Serag, Gastroenterology; 142:1264-73 (2012)). Treatment for liver cancer typically includes surgery and ablation, but for advanced or end stage patients, such treatments are often ineffective.

### SUMMARY

This document provides methods and materials for treating a mammal having cancer by converting cancer cells within the mammal into non-cancerous cells. For example, one or more nucleic acids encoding a transcription factor (e.g., a neuronal transcription factor or a liver transcription factor) can be used to convert one or more cancer cells within the mammal into non-cancerous cells.

A hallmark of many cancers is the presence of dedifferentiated cancer cells. As described herein, delivering nucleic acid designed to express a transcription factor (e.g., a neuronal transcription factor or a liver transcription factor) to cells within a mammal can convert cancer cells into non-cancerous cells (e.g., terminally differentiated, non-dividing cells) within the mammal. As demonstrated herein, delivering nucleic acid designed to express a neuronal transcription factor (e.g., nucleic acid designed to express a neurogenic differentiation factor 1 (NeuroD1) polypeptide, nucleic acid designed to express a neurogenin-2 (Neurog2) polypeptide, or nucleic acid designed to express an achaete-scute homolog 1 (Ascl1) polypeptide) to human GBM cells can convert the human GBM cells to non-cancerous neurons. The converted neurons can express neuron-specific markers, can have functional synaptic networks, and can have active electrophysiological properties. The converted neurons also can exhibit downregulated signaling pathways related to cancer progression (e.g., as compared to the GBM cells prior to conversion). The in vivo conversion of GBM cells to neurons can reduce cancer cell proliferation and/or can decrease the rate of astrogliosis. Also as demonstrated herein, delivering nucleic acid designed to express a liver transcription factor (e.g., delivering nucleic acid designed to express a hepatocyte nuclear factor 4A (HNF4A) polypeptide, nucleic acid designed to express a forkhead box protein (Foxa2) polypeptide, and/or nucleic acid designed to express a GATA binding protein (GATA4) polypeptide) to human liver cancer cells can convert the human liver cancer cells to non-cancerous liver cells (hepatocytes). The converted hepatocytes can have decreased proliferation, can have decreased expression of the liver cancer markers alpha fetoprotein (AFP), and/or can express epithelial-specific markers such as the epithelial cell surface molecule E-cadherin.

Having the ability to convert cancer cells into non-cancerous cells within a living mammal using the methods and materials described herein provides clinicians and patients (e.g., cancer patients) with an effective approach to treat cancer. For example, the in vivo conversion of cancer cells into non-cancerous cells can be used to control proliferation of cancer cells in the absence of traditional cancer therapy. In such cases, a cancer patient can avoid common side effects caused by traditional cancer therapies.

In general, one aspect of this document features a method for treating a mammal having a cancer. The method comprises (or consists essentially of or consists of) administering nucleic acid encoding one or more transcription factors to cancer cells within the mammal, wherein the one or more transcription factors are expressed by the cancer cells, and wherein the one or more transcription factors convert the cancer cells into non-cancerous cells within the mammal, thereby reducing the number of cancer cells within the mammal. The mammal can be a human. The cancer can be a glioma. The one or more transcription factors can be one or more neuronal transcription factors. The one or more neuronal transcription factors can be selected from the group consisting of a neurogenic differentiation factor 1 (NeuroD1) polypeptide, a neurogenin-2 (Neurog2) polypeptide, and an achaete-scute homolog 1 (Ascl1) polypeptide. The one or more neuronal transcription factors can comprise a NeuroD1 polypeptide, a Neurog2 polypeptide, and an Ascl1 polypeptide. The non-cancerous cells can be neurons. The neurons can be FoxG1-positive forebrain neurons. The cancer can be a liver cancer. The liver cancer can be a hepatocellular carcinoma. The one or more transcription factors can be liver transcription factors. The one or more liver transcription factors can be selected from the group consisting of a hepatocyte nuclear factor 4A (HNF4A) polypeptide, a forkhead box protein (Foxa2) polypeptide, and a GATA binding protein (GATA4) polypeptide. The one or more liver transcription factors can comprise a HNF4A polypeptide, a Foxa2 polypeptide, and a GATA4 polypeptide. The non-cancerous cells can be hepatocytes. The hepatocytes can be hepatocytes that secrete a liver enzyme. The liver enzyme can be albumin. The nucleic acid encoding the one or more transcription factors can be administered to the cancer cells in the form of a viral vector. The viral vector can be a retroviral vector. The viral vector can be a lentiviral vector. The nucleic acid encoding each of the one or more transcription factors can be operably linked to a promoter sequence. The administration of the nucleic acid encoding the one or more transcription factors can comprise a direct injection into a tumor of the mammal. The administration of the nucleic acid encoding the one or more transcription factors can comprise an intraperitoneal, intramuscular, intravenous, intrathecal, intracerebral, intraparenchymal, intratumoral, intranasal, or oral administration. The method can comprise, prior to the administering step, identifying the mammal as having the cancer.

In another aspect, this document features the use of a composition comprising (or consisting essentially of or consisting of) nucleic acid encoding one or more transcription factors to treat cancer according to a method comprises (or consists essentially of or consists of) administering nucleic acid encoding one or more transcription factors to cancer cells within the mammal, wherein the one or more transcription factors are expressed by the cancer cells, and wherein the one or more transcription factors convert the cancer cells into non-cancerous cells within the mammal, thereby reducing the number of cancer cells within the mammal. The mammal can be a human. The cancer can be a glioma. The one or more transcription factors can be one or more neuronal transcription factors. The one or more neuronal transcription factors can be selected from the group consisting of a neurogenic differentiation factor 1 (NeuroD1) polypeptide, a neurogenin-2 (Neurog2) polypeptide, and an achaete-scute homolog 1 (Ascl1) polypeptide. The one or more neuronal transcription factors can comprise a NeuroD1 polypeptide, a Neurog2 polypeptide, and an Ascl1 polypeptide. The non-cancerous cells can be neurons. The neurons can be FoxG1-positive forebrain neurons. The cancer can be a liver cancer. The liver cancer can be a hepatocellular carcinoma. The one or more transcription factors can be liver transcription factors. The one or more liver transcription factors can be selected from the group consisting of a hepatocyte nuclear factor 4A (HNF4A) polypeptide, a forkhead box protein (Foxa2) polypeptide, and a GATA binding protein (GATA4) polypeptide. The one or more liver transcription factors can comprise a HNF4A polypeptide, a Foxa2 polypeptide, and a GATA4 polypeptide. The non-cancerous cells can be hepatocytes. The hepatocytes can be hepatocytes that secrete a liver enzyme. The liver enzyme can be albumin. The nucleic acid encoding the one or more transcription factors can be administered to the cancer cells in the form of a viral vector. The viral vector can be a retroviral vector. The viral vector can be a lentiviral vector. The nucleic acid encoding each of the one or more transcription factors can be operably linked to a promoter sequence. The administration of the nucleic acid encoding the one or more transcription factors can comprise a direct injection into a tumor of the mammal. The administration of the nucleic acid encoding the one or more transcription factors can comprise an intraperitoneal, intramuscular, intravenous, intrathecal, intracerebral, intraparenchymal, intratumoral, intranasal, or oral administration. The method can comprise, prior to the administering step, identifying the mammal as having the cancer.

In another aspect, this document features a composition comprising (or consisting essentially of or consisting of) nucleic acid encoding one or more transcription factors to treat cancer according to a method comprises (or consists essentially of or consists of) administering nucleic acid encoding one or more transcription factors to cancer cells within the mammal, wherein the one or more transcription factors are expressed by the cancer cells, and wherein the one or more transcription factors convert the cancer cells into non-cancerous cells within the mammal, thereby reducing the number of cancer cells within the mammal. The mammal can be a human. The cancer can be a glioma. The one or more transcription factors can be one or more neuronal transcription factors. The one or more neuronal transcription factors can be selected from the group consisting of a neurogenic differentiation factor 1 (NeuroD1) polypeptide, a neurogenin-2 (Neurog2) polypeptide, and an achaete-scute homolog 1 (Ascl1) polypeptide. The one or more neuronal transcription factors can comprise a NeuroD1 polypeptide, a Neurog2 polypeptide, and an Ascl1 polypeptide. The non-cancerous cells can be neurons. The neurons can be FoxG1-positive forebrain neurons. The cancer can be a liver cancer. The liver cancer can be a hepatocellular carcinoma. The one or more transcription factors can be liver transcription factors. The one or more liver transcription factors can be selected from the group consisting of a hepatocyte nuclear factor 4A (HNF4A) polypeptide, a forkhead box protein (Foxa2) polypeptide, and a GATA binding protein (GATA4) polypeptide. The one or more liver transcription factors can comprise a HNF4A polypeptide, a Foxa2 polypeptide, and a GATA4 polypeptide. The non-cancerous cells can be hepatocytes. The hepatocytes can be hepatocytes that secrete a liver enzyme. The liver enzyme can be albumin. The nucleic acid encoding the one or more transcription factors can be administered to the cancer cells in the form of a viral vector. The viral vector can be a retroviral vector. The viral vector can be a lentiviral vector. The nucleic acid encoding each of the one or more transcription factors can be operably linked to a promoter sequence. The administration of the nucleic acid encoding the one or more transcription factors can comprise a direct injection into a tumor of the mammal. The administration of the nucleic acid encoding the one or more transcription factors can comprise an intraperitoneal, intramuscular, intravenous, intrathecal, intracerebral, intraparenchymal, intratumoral, intranasal, or oral administration. The method can comprise, prior to the administering step, identifying the mammal as having the cancer.

In another aspect, this document features the use of nucleic acid encoding one or more transcription factors in the manufacture of a medicament to treat cancer according to a method comprises (or consists essentially of or consists of) administering nucleic acid encoding one or more transcription factors to cancer cells within the mammal, wherein the one or more transcription factors are expressed by the cancer cells, and wherein the one or more transcription factors convert the cancer cells into non-cancerous cells within the mammal, thereby reducing the number of cancer cells within the mammal. The mammal can be a human. The cancer can be a glioma. The one or more transcription factors can be one or more neuronal transcription factors. The one or more neuronal transcription factors can be selected from the group consisting of a neurogenic differentiation factor 1 (NeuroD1) polypeptide, a neurogenin-2 (Neurog2) polypeptide, and an achaete-scute homolog 1 (Ascl1) polypeptide. The one or more neuronal transcription factors can comprise a NeuroD1 polypeptide, a Neurog2 polypeptide, and an Ascl1 polypeptide. The non-cancerous cells can be neurons. The neurons can be FoxG1-positive forebrain neurons. The cancer can be a liver cancer. The liver cancer can be a hepatocellular carcinoma. The one or more transcription factors can be liver transcription factors. The one or more liver transcription factors can be selected from the group consisting of a hepatocyte nuclear factor 4A (HNF4A) polypeptide, a forkhead box protein (Foxa2) polypeptide, and a GATA binding protein (GATA4) polypeptide. The one or more liver transcription factors can comprise a HNF4A polypeptide, a Foxa2 polypeptide, and a GATA4 polypeptide. The non-cancerous cells can be hepatocytes. The hepatocytes can be hepatocytes that secrete a liver enzyme. The liver enzyme can be albumin. The nucleic acid encoding the one or more transcription factors can be administered to the cancer cells in the form of a viral vector. The viral vector can be a retroviral vector. The viral vector can be a lentiviral vector. The nucleic acid encoding each of the one or more transcription factors can be operably linked to a promoter sequence. The administration of the nucleic acid encoding the one or more transcription factors can comprise a direct injection into a tumor of the mammal. The administration of the nucleic acid encoding the one or more transcription factors can comprise an intraperitoneal, intramuscular, intravenous, intrathecal, intracerebral, intraparenchymal, intratumoral, intranasal, or oral administration. The method can comprise, prior to the administering step, identifying the mammal as having the cancer.

Unless otherwise defined, all technical and scientific terms used herein have the same meaning as commonly understood by one of ordinary skill in the art to which this invention pertains, as exemplified by various art-specific dictionaries. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more aspects of the invention are set forth in the accompanying drawings and the description below. Other features, objects, and advantages of the invention will be apparent from the description and drawings, and from the claims.

### DESCRIPTION OF THE DRAWINGS

FIG. 1. Characterization of human glioblastoma cell lines. Representative images of a series of markers (which stained red) to characterize U251 and U118 human glioblastoma cells. Red boxes (marked with *) indicate high level of immunopositive markers. Scale bars, 50 μm. GFAP and S100β, astrocyte markers; Tuj1 and DCX, immature neuronal markers; Sox2 and Nestin, neural progenitor markers; Olig2, oligodendrocyte marker; Ki67, cell proliferation marker; EGFR, cancer marker.

FIG. 2 Confirmation of the overexpression of neural transcription factor Neurog2, NeuroD1 or Ascl1 in human glioblastoma cells. A, Representative images showing the overexpression of Neurog2, NeuroD1, or Ascl1 in U251 human glioblastoma cells through immunostaining. Scale bar, 20 μm. B, Hierarchical clustering and heat map of real-time qPCR analyses showing the transcriptional changes of different neural transcription factors in U251 GBM cells. Note a huge increase of mRNA level in U251 GBM cells after viral infection by Neurog2, NeuroD1 or Ascl1. Data were normalized to GFP control virus-infected U251 cells and represented as mean value. Samples were collected at 20 days post infection (dpi). n=3 batches of cultures.

FIG. 3. Rapid induction of neuron-like cells from human glioblastoma cells by Neurog2 and NeuroD1. A, Immunostaining of immature neuronal markers Doublecortin (DCX, red) and β3-tubulin (Tuj1, magenta) in U251 human GBM cells infected by Neurog2-GFP, NeuroD1-GFP or Ascl1-GFP retroviruses at 6 dpi. Scale bar, 50 μm. B-C, Quantitative analyses of neuronal conversion at 6 dpi. Note that Neurog2 showed a significant increase in the percentage of DCX⁺ cells (GFP, 0; Neurog2, 12.6%±2.0%; NeuroD1, 1.6%±0.4%; Ascl1, 0; B), and Tuj1⁺ cells (GFP, 0; Neurog2, 46.1%±3.2%; NeuroD1, 20.5%±4.9%; Ascl1, 2.6%±0.7%; C), followed by NeuroD1 at early stage of viral infection. The Ascl1 conversion efficiency is the lowest among the three neural transcription factors tested. Data are represented as mean±SEM, and analyzed by one-way ANOVA followed with Dunnett's test. **, p<0.01; ***, p<0.001; n>200 cells from triplicate cultures.

FIG. 4. Single neuronal transcription factor Neurog2, NeuroD1, or Ascl1 converts human glioblastoma cells into neurons. A-B, Retroviral expression of Neurog2-GFP, NeuroD1-GFP, or Ascl1-GFP in U251 human glioblastoma cells led to a large number of neuronal cells compared with GFP alone (top row). Neurog2-, NeuroD1-, or Ascl1-converted cells were immunopositive for immature neuronal markers (A; DCX; Tuj1) at 20 days post infection (dpi), and mature neuronal markers (B; MAP2; NeuN) at 30 dpi. Scale bars, 50 μm. C-D, Quantitative analyses of conversion efficiency at 20 dpi (C) and 30 dpi (D). ** p<0.01; *** p<0.001; one-way ANOVA followed with Dunnett's test; n≥200 cells from triplicate cultures. E, Time course of DCX transcriptional activation with Neurog2, NeuroD1, or Ascl1 overexpression in U251 cells revealed by real-time qPCR. Data were normalized to GFP control and represented as mean±SEM. n=3 batches.

FIG. 5. Induction of neuron-like cells in U118 human glioblastoma cells via combination of Neurog2 overexpression and small molecule treatment. A-D, U118 cells were converted into neuron-like cells (DCX, which stained in cyan) when Neurog2 was overexpressed together with small molecule treatment (Core: 5 μM DAPT, 1.5 μM CHIR99021, 5 μM SB431542, 0.25 μM LDN193189). Samples were collected at 18 dpi with 12-day drug treatment.

FIG. 6. Characterization of the converted neurons from human GBM cells. A-D, Representative images showing the immunostaining of neuronal subtype markers. Most of the Neurog2-, NeuroD1-, and Ascl1-converted neurons (DCX in A; and MAP2 in B) were immunopositive for hippocampal neuron marker Prox1 (A) and forebrain neuron marker FoxG1 (B). Furthermore, Neurog2-, NeuroD1-, and Ascl1-converted neurons (DCX in C) were largely VGluT1+ (C), while some of the Ascl1-converted neurons (DCX in D) were also GABA+ (D). E-H, Quantitative analyses of converted neurons from human GBM cells. Samples were at 20 dpi. Scale bars=50 μm. Data are represented as mean±SEM. n≥200 cells from triplicate cultures.

FIG. 7. Further characterization of the neuronal identity among the human GBM cell-converted neurons. A-B, Representative images showing the immunostaining signal for cortical neuron marker Ctip2 (A) or Tbr1 (B) after viral infection by Neurog2, NeuroD1, or Ascl1 among U251 human glioblastoma cells at 20 dpi. Scale bars, 50

FIG. 8. Comparison with the human astrocyte-converted neurons after infection by Neurog2, NeuroD1, or Ascl1. A, Representative images showing that the majority of human astrocyte-converted neurons induced by Neurog2, NeuroD1 or Ascl1 were immunopositive for hippocampal neuron marker Prox1 and forebrain marker FoxG1. Much less Ascl1-converted neurons were Ctip2⁺. Scale bars, 20 μm. B, Quantitative analyses of Neurog2-, NeuroD1- and Ascl1-converted neurons from human cortical astrocytes (HA1800 cells, ScienCell, San Diego, USA). Prox1⁺/MAP2⁺: Neurog2, 85.4%±3.4%; NeuroD1, 89.2%±3.3%; Ascl1, 85.0%±3.7%. FoxG1⁺/MAP2⁺: Neurog2, 92.6%±3.8%; NeuroD1, 85.1%±2.7%; Ascl1, 85.7%±4.8%. Ctip2⁺/MAP2⁺: Neurog2, 46.6%±5.1%; NeuroD1, 61.1%±2.8%; Ascl1, 14.0%±5.4%. Samples were at 30 dpi. Data are represented as mean±SEM. n>50 cells from triplicate cultures.

FIG. 9. Fate change from glioblastoma cells to neurons induced by Neurog2 overexpression. A, Downregulation of astrocyte markers vimentin and GFAP in Neurog2-converted neurons (bottom row) compared to the control U251 glioblastoma cells expressing GFP alone (top row). Samples were at 20 dpi. B-C, Representative images showing the gap junctions (Connexin 43) among U251 GBM cells overexpressing GFP alone (top row) or Neurog2-GFP (bottom row). Quantified data (C) showing a significant reduction of Connexin 43 intensity in Neurog2 group compared with control GFP group. Samples were at 20 dpi. n>60 from triplicate cultures. D, Representative images illustrating a growth cone depicted by GAP43 and phalloidin in U251 cells overexpressing Neurog2 at 6 dpi. E-H, Distribution and morphological changes of mitochondria (MitoTracker) and the Golgi apparatus (GM130) during neuronal conversion of U251 cells. Quantified data showing changes of MitoTracker intensity (F) and the Golgi apparatus size reflected by GM130 covered area (H) after Neurog2 expression at 30 dpi. n>150 from triplicate cultures. Scale bars, 20 μm in (A), (B), and (D); 10 μm in (E) and (G). Data are represented as mean±SEM and analyzed by Student's t-test. *=p<0.05; ***=p<0.001.

FIG. 10. Neuronal conversion of human glioblastoma cells inhibits proliferation. A, Representative images examining cell proliferation through BrdU immunostaining in U251 human glioblastoma cells expressing GFP, Neurog2-GFP, NeuroD1-GFP, or Ascl1-GFP. Cell cultures were incubated in 10 mM BrdU for 24 hours before immunostaining at 7 dpi. Scale bars=50 μm. B, Quantitative analyses of proliferative cells (BrdU+ cells/total infected cells) during neuronal conversion of U251 cells. Data were analyzed by one-way ANOVA followed with Dunnett's test. ***=p<0.001; n>200 cells from triplicate cultures. C-D, GSK3β expression level examined by western blot in U251 GBM cells overexpressing GFP alone or Neurog2-GFP. Data were normalized to GFP control (D). Samples were collected at 20 dpi. n=3 batches. E, GSK3β immunostaining in U251 GBM cells upon GFP or Neurog2-GFP overexpression (GFP) at 20 dpi. Note a significant increase of GSK3β signal in Neurog2-converted neurons. Scale bars=50 μm. F, Quantitative analyses of GSK3β immunostaining intensity during neuronal conversion of U251 cells. Samples were collected at 20 dpi. Data were analyzed by Student's t-test. ***=p<0.001; n=6 batches. Data are represented as mean±SEM.

FIG. 11. Examination of autophagy/lysosomes during neuronal conversion of human GBM cells. A, Representative images illustrating the distribution and morphological changes of autophagy/lysosomes (ATG5, which stained red) during neuronal conversion of U251 cells. B-C, Quantified data of ATG5 area (B) and intensity (C) in infected U251 cells at 30 dpi. n≥150 cells from triplicate cultures. Scale bars, 10 Data are represented as mean±SEM, and analyzed by Student's t-test. * p<0.05; *** p<0.001.

FIG. 12. Functional analyses of human glioblastoma cell-converted neurons. A, Robust synaptic puncta (SV2) were detected along the dendrites (MAP2, cyan) in Neurog2-converted neurons from U251 human glioblastoma cells. Scale bars=20 μm. B-C, Representative traces (B) showing Na+ and K+ currents recorded from Neurog2-converted neurons, with quantitative analyses shown in (C). D-E, Whole-cell patch-clamp recordings revealed action potentials firing from Neurog2-converted neurons (D), with a pie chart indicating the fraction of cells firing single (dark grey, E), repetitive (light grey, E) or no action potentials (black, E). Samples were at 30 dpi. n≥20 from triplicate cultures.

FIG. 13. Inhibition of GSK3β affects neuronal conversion of human GBM cells. A, Immunostaining of GSK3β (which stained magenta) in Neurog2-converted neurons (DCX, which stained red) from U251 human GBM cells at 20 dpi. Scale bars, 50 μm. B-C, Quantitative analyses of GSK3β intensity (B) and neuronal conversion efficiency (C) after inhibiting GSK3β by CHIR99021 (5 μM) or TWS119 (10 μM) for 20 days. Data are represented as mean±SEM, and analyzed by Student's t-test. n>3 repeats.

FIG. 14. Investigation of cancer makers in Neurog2-converted neurons from human glioblastoma cells. A-B, Immunostaining of IL13Ra2 (which stained red, A) in Neurog2-converted neurons from U251 human glioblastoma cells at 20 dpi. Quantified in panel (B). Scale bar, 50 μm. C-D, Immunostaining of EGFR (which stained red, C) during neuronal conversion of U251 cells. Samples were collected at 20 dpi. Scale bar, 20 μm. Data are represented as mean±SEM, and were analyzed by Student's t-test. n>40 cells from triplicate samples.

FIG. 15. In vivo neuronal conversion of human glioblastoma cells in a xenograft mouse model. A, Representative images illustrating the transplanted human U251 GBM cells (mixed with Neurog2-GFP retroviruses) in the brain of Rag1^{-/-} immunodeficient mice at one month post transplantation. Note that U251 cells expressed a high level of vimentin, and Neurog2-GFP infected U251 cells were immunopositive for immature neuronal marker DCX. B, Quantitative analyses of conversion efficiency at 1-month post transplantation. Data are represented as mean±SEM and analyzed by Student's t-test. ***=p<0.001; n=3 animals. Note that the in vivo conversion efficiency was also very high (˜90%). C, High magnification images showing that most of the transplanted U251 cells (Vimentin) infected by Neurog2-GFP (bottom row) retroviruses were converted into neurons (DCX) at 1-week post transplantation. D-E, Further characterization of Neurog2-converted neurons in vivo as shown by neuronal marker Tuj1 and hippocampal neuronal marker Prox1 at 1-month post transplantation of U251 human glioblastoma cells (labeled by Vimentin and Human Nuclei). Scale bars=200 μm in (A) and 20 μm in (C)-(E).

FIG. 16. Inhibition of cell proliferation and reduction of astrogliosis after in vivo neuronal conversion of glioblastoma cells. A-B, Representative images (A) and quantitative analyses (B) of proliferating U251 GBM cells (Ki67+) at 7 days post transplantation. Note a significant reduction of cell proliferation in Neurog2 group. n=4 animals. C-D, Reduction of reactive astrocytes (labeled by LCN2) in the Neurog2-infected regions (D), compared to the contralateral GFP-infected regions (C). Samples were at three weeks post transplantation. E, Quantitative analyses of LCN2-covered area at three weeks post U251 cell transplantation (infected by Neurog2-GFP or GFP retroviruses). n=5 animals. Scale bars=50 μm. Data are represented as mean±SEM and analyzed by Student's t-test. **=p<0.01; ***=p<0.001.

FIG. 17. Resident microglia and blood vessel distribution during in vivo neuronal conversion of glioblastoma cells. A-B, Examination of resident microglia (Iba1, red) at 3 weeks post U251 GBM cell transplantation with Neurog2 or GFP control virus-infection. Quantitative analyses of Iba1 mean intensity in panel (B). n=3 animals. Scale bar, 100 μm. C-D, Representative images showing blood vessel distribution (Lytic, which stained red) at 3 weeks post U251 GBM cell transplantation with Neurog2 or GFP control virus-infection. Quantitative analyses of Lytic-covered area in panel (C). Data are represented as mean±SEM, and analyzed by Student's t-test. n=5 animals. Scale bar, 100 μm.

FIG. 18. Transduction of liver tumor cell line HepG2 by liver transcription factors Foxa2, HNF4A, and GATA4. A, Transduced cells grown on glass coverslips were fixed with paraformaldehyde and bound with a mixture of chicken anti-GFP plus goat anti-Foxa2 or chicken anti-GFP plus goat anti-HNF4A or chicken anti-GFP plus goat anti-GATA4 antibodies. Secondary antibodies chicken-specific Alexa Fluor 488 and goat-specific Alexa Fluor 594 were used for detection. Fluorescence was visualized with Zeiss LSM800 confocal microscope. B, Transduced cells grown in 12-well plate were harvested, and cell lysates were processed for SDS-PAGE and analyzed by western blotting with a mouse monoclonal anti-GFP antibody. C, D, E, Cell lysates were fractionated by SDS-PAGE and processed for immunoblotting with a goat polyclonal anti-Foxa2, goat polyclonal anti-HNF4A or goat polyclonal anti-GATA4 antibody, respectively.

FIG. 19. Transduction of GATA4 increases endogenous Foxa2 expression level. A, Foxa2, HNF4A, GATA4, or GFP transduced HepG2 cells were harvested, and cell lysates were fractionated by SDS-PAGE and analyzed by immunoblotting with a mixture of goat polyclonal anti-Foxa2 and mouse monoclonal anti-HNF4A antibodies. A rabbit GAPDH polyclonal antibody was used as internal loading control. B, The above cell lysates were fractionated by SDS-PAGE and analyzed by immunoblotting with a mixture of goat polyclonal anti-GATA4 and rabbit polyclonal anti-GAPDH antibodies.

FIG. 20. In vitro and in vivo cell proliferation of Foxa2, GATA4, HNF4A, or GFP transduced cell lines. A, In vitro proliferation curve of Foxa2, GATA4, HNF4A, or GFP transduced cell lines. Equal amount of Foxa2, GATA4, HNF4A, or GFP transduced cells were seeded in 12-well plate. At different time points, cells were fixed and stained with crystal violet. The stained crystal violet was extracted by acetic acid, and the optical density of each extraction was read by a microplate reader. The volume of optical density represent the cells number grown in each well. Each value represent three individual experiments. B, Tumor growth curves of Foxa2, GATA4, HNF4A, or GFP transduced cell lines. Foxa2, GATA4, HNF4A, or GFP transduced cell lines were subcutaneously injected into the flank of nude mice. The tumors were monitored every four days by measuring tumor size using caliper. The tumor volume was calculated by the following formula: V=width×length×length×0.5.

FIG. 21. Expression and secretion of albumin from Foxa2, GATA4, HNF4A, or GFP transduced cells. A, Transduced cells grown on glass coverslips were bound with a mixture of chicken anti-GFP plus goat anti-albumin antibodies. Secondary antibodies chicken-specific Alexa Fluor 488 and goat-specific Alexa Fluor 594 were used for detection. Fluorescence was visualized with Zeiss LSM800 confocal microscope as described for FIG. 18A. B, Albumin expressed in Foxa2, GATA4, HNF4A, or GFP transduced cells were detected by western blot with a goat polyclonal anti-albumin antibody. Rabbit polyclonal anti-GAPDH antibody was used to show internal loading control. C, Relatively albumin production was calculated as the amount of albumin protein detected in Foxa2, GATA4, or HNF4A transduced cells normalized to the amount of albumin obtained with GFP transduced cells. Results were from three independent experiments. D, Albumin concentration secreted into culture medium of Foxa2, GATA4, HNF4A, or GFP transduced cells. Albumin produced in Foxa2, GATA4, HNF4A, or GFP transduced cells were secreted into culture medium. The concentration of albumin in culture medium were measured by ELISA according to the ELISA kit instructions. The albumin concentration were obtained by comparison with a standard curve provided in the kit.

FIG. 22. Liver cancer marker alpha fetoprotein (AFP) expression in Foxa2, GATA4, HNF4A, or GFP transduced cells. A, Foxa2, GATA4, HNF4A, or GFP transduced cells were grown on coverslips, fixed, and stained with a mixture of chicken anti-GFP plus rabbit anti-AFP. A secondary antibodies mixture of chicken-specific Alexa Fluor 488 and rabbit-specific Alexa Fluor 594 was used for detection. Fluorescence was visualized with Zeiss LSM800 confocal microscope as described for FIG. 18A. B, Foxa2, GATA4, HNF4A, or GFP transduced cells were lysed and fractionated by SDS-PAGE and probed with a rabbit polyclonal anti-AFP for immunoblotting analysis. A rabbit GAPDH polyclonal antibody was used as internal loading control. Relatively AFP level was calculated as the amount of AFP detected in Foxa2, GATA4, or HNF4A transduced cells normalized to the amount of AFP obtained with GFP transduced cells. Results were from three independent experiments. C, AFP levels in tumors formed by GFP, HNF4A, or GATA4 transduced cells. Tumor sections were permeabilized with Triton X-100, followed by incubation with primary antibodies chicken anti-GFP plus rabbit anti-AFP. A secondary antibodies mixture of chicken-specific Alexa Fluor 488 and rabbit-specific Alexa Fluor 594 were used for detection. Fluorescence was visualized with Zeiss LSM800 confocal microscope. D, Xenografted tumors of GATA4, HNF4A, or GFP cell lines were freshly collected and lysed. The lysates were separated by SDS-PAGE gel, and were probed with AFP, GATA4, and GFP antibodies. Actin was shown as internal loading control. Relatively AFP level was calculated as the amount of AFP detected in GATA4 or HNF4A transduced cells normalized to the amount of AFP obtained with GFP transduced cells as described for panel B. Results were from three independent experiments.

FIG. 23. Over-expression of GATA4, Foxa2, or HNF4A leads to an increase of membranous E-cadherin. A, Foxa2, GATA4, HNF4A, or GFP transduced cells were stained with a mixture of chicken anti-GFP plus rabbit anti-E-cadherin. A secondary antibodies mixture of chicken-specific Alexa Fluor 488 and rabbit-specific Alexa Fluor 594 were used for detection. Fluorescence was visualized with Zeiss LSM800 confocal microscope as described for FIG. 18A. B, Lysed Foxa2, GATA4, HNF4A, or GFP transduced cells were fractionated by SDS-PAGE and probed with a rabbit polyclonal anti-Ecadherin for western blot analysis. A rabbit GAPDH polyclonal antibody was used as internal loading control. Relative AFP levels were calculated as the amount of E-cadherin detected in Foxa2, GATA4, or HNF4A transduced cells normalized to the amount of E-cadherin obtained with GFP transduced cells. Results were from three independent experiments. C, Immunofluorescent images of E-cadherin showing increased E-cadherin in GATA4 or HNF4A transduced cells formed tumors compared to GFP tumor. D, Tumor samples of GATA4, HNF4A, or GFP transduced cells were analysed quantitatively by western blot with anti-E-cadherin antibody. E-cadherin expressed more intensely with a measurable 2-fold higher intensity, as shown in GATA4 comparing with GFP expressing tumor cells.

FIG. 24. Expression and redistribution of beta-catenin in GATA4 overexpressed cell line. A, GATA4 or GFP transduced cells were stained with rabbit anti-beta-catenin antibody, and immunofluorescent images of beta-catenin were visualized with a microscope. Beta-catenin in GATA4 cells was distributed to cell surface. B, Western blot analysis of Foxa2, GATA4, HNF4A, or GFP transduced cells with beta-catenin antibody and relative beta-catenin levels detected in Foxa2, GATA4, HNF4A, or GFP transduced cells. Results were from three independent experiments. C, Immunofluorescent images of beta-catenin showing beta-catenin in GATA4 transduced cells formed tumors compared to GFP tumor. D, Tumor samples of GATA4, HNF4A, or GFP transduced cells were analysed by western blot with an anti-beta-catenin antibody. Beta-catenin expressed in tumors of GATA4, HNF4A, or GFP transduced cells was calculated.

FIG. 25. Vimentin expression in tumors of GATA4, HNF4A, and GFP transduced cells. Western blot analysis of vimentin expressed in tumors of GATA4, HNF4A, or GFP transduced cells with anti-vimentin antibody revealed vimentin levels decreased in GATA4 transduced cells formed tumors compared to GFP tumor. Decreased vimentin was calculated by comparison of the amount of vimentin detected in GATA4 transduced cells normalized to the amount of obtained with GFP transduced cells.

FIG. 26. Amino acid sequence of a representative NeuroD1 polypeptide (SEQ ID NO:1).

FIG. 27. Amino acid sequence of a representative Neurog2 polypeptide (SEQ ID NO:2).

FIG. 28. Amino acid sequence of a representative Ascl1 polypeptide (SEQ ID NO:3).

FIG. 29. Amino acid sequence of a representative HNF4A polypeptide (SEQ ID NO:4).

FIG. 30. Amino acid sequence of a representative Foxa2 polypeptide (SEQ ID NO:5).

FIG. 31. Amino acid sequence of a representative GATA4 polypeptide (SEQ ID NO:6).

### DETAILED DESCRIPTION

As used herein, the singular form “a,” “an,” and “the” include plural references unless the context clearly dictates otherwise.

This document provides methods and materials for treating a mammal having cancer. For example, nucleic acid encoding one or more transcription factors, or one or more transcription factors themselves, can be used to treat a mammal having cancer. In some cases, treating a mammal having cancer as described herein can include converting cancer cells within the mammal into non-cancerous cells (e.g., functional cells or near normal cells) within the mammal. In some cases, treating a mammal having cancer as described herein can have a conversion efficacy of, for example, 10, 20, 30, 40, 50, 60, 70, 80, 90, 95, or more percent. In some cases, treating a mammal having cancer as described herein can have a conversion efficacy from 10 to 100 percent, such as from 10 to 15 percent, from 10 to 20 percent, from 10 to 25 percent, from 15 to 20 percent, from 15 to 25 percent, from 15 to 30 percent, from 20 to 25 percent, from 20 to 30 percent, from 20 to 35 percent, from 25 to 30 percent, from 25 to 35 percent, from 25 to 40 percent, from 30 to 35 percent, from 30 to 40 percent, from 35 to 45 percent, from 35 to 50 percent, from 40 to 45 percent, from 40 to 50 percent, from 40 to 55 percent, from 45 to 50 percent, from 45 to 55 percent, from 45 to 60 percent, from 50 to 55 percent, from 50 to 60 percent, from 50 to 65 percent, from 55 to 60 percent, from 55 to 65 percent, from 55 to 70 percent, from 60 to 65 percent, from 60 to 70 percent, from 60 to 75 percent, from 65 to 70 percent, from 65 to 75 percent, from 65 to 80 percent, from 70 to 75 percent, from 70 to 80 percent, from 70 to 85 percent, from 75 to 80 percent, from 75 to 85 percent, from 75 to 90 percent, from 80 to 85 percent, from 80 to 90 percent, from 80 to 95 percent, from 85 to 90 percent, from 85 to 95 percent, from 85 to 100 percent, from 90 to 95 percent, from 90 to 100 percent, or from 95 to 100 percent. For example, treating a mammal having cancer as described herein can be effective to convert, for example, 10, 20, 30, 40, 50, 60, 70, 80, 90, 95, or more percent of cancer cells within the mammal into non-cancerous cells (e.g., functional cells or near-normal cells). In some cases, treating a mammal having cancer as described herein can be effective to convert cancer cells within the mammal into non-cancerous cells from 10 to 100 percent, such as from 10 to 15 percent, from 10 to 20 percent, from 10 to 25 percent, from 15 to 20 percent, from 15 to 25 percent, from 15 to 30 percent, from 20 to 25 percent, from 20 to 30 percent, from 20 to 35 percent, from 25 to 30 percent, from 25 to 35 percent, from 25 to 40 percent, from 30 to 35 percent, from 30 to 40 percent, from 35 to 45 percent, from 35 to 50 percent, from 40 to 45 percent, from 40 to 50 percent, from 40 to 55 percent, from 45 to 50 percent, from 45 to 55 percent, from 45 to 60 percent, from 50 to 55 percent, from 50 to 60 percent, from 50 to 65 percent, from 55 to 60 percent, from 55 to 65 percent, from 55 to 70 percent, from 60 to 65 percent, from 60 to 70 percent, from 60 to 75 percent, from 65 to 70 percent, from 65 to 75 percent, from 65 to 80 percent, from 70 to 75 percent, from 70 to 80 percent, from 70 to 85 percent, from 75 to 80 percent, from 75 to 85 percent, from 75 to 90 percent, from 80 to 85 percent, from 80 to 90 percent, from 80 to 95 percent, from 85 to 90 percent, from 85 to 95 percent, from 85 to 100 percent, from 90 to 95 percent, from 90 to 100 percent, or from 95 to 100 percent.

In some cases, nucleic acid designed to express one or more transcription factors (or the one or more transcription factors themselves) can be administered to a mammal in need thereof (e.g., a mammal having cancer) to reduce the size of the cancer in the mammal (e.g., reduce the number of cancer cells in the mammal and/or the volume of one or more tumors in the mammal). For example, nucleic acid designed to express one or more neuronal transcription factors (or the one or more neuronal transcription factors themselves) can be administered to a mammal (e.g., a human) having brain cancer as described herein to reduce the size of the brain cancer by, for example, 10, 20, 30, 40, 50, 60, 70, 80, 90, 95, or more percent. In another example, nucleic acid designed to express one or more liver transcription factors (or the one or more liver transcription factors themselves) can be administered to a mammal (e.g., a human) having liver cancer as described herein to reduce the size of the liver cancer by, for example, 10, 20, 30, 40, 50, 60, 70, 80, 90, 95, or more percent. In some cases, nucleic acid designed to express one or more liver transcription factors (or the one or more liver transcription factors themselves) can be administered to a mammal (e.g., a human) having liver cancer as described herein to reduce the size of the liver cancer from 10 to 100 percent, such as from 10 to 15 percent, from 10 to 20 percent, from 10 to 25 percent, from 15 to 20 percent, from 15 to 25 percent, from 15 to 30 percent, from 20 to 25 percent, from 20 to 30 percent, from 20 to 35 percent, from 25 to 30 percent, from 25 to 35 percent, from 25 to 40 percent, from 30 to 35 percent, from 30 to 40 percent, from 35 to 45 percent, from 35 to 50 percent, from 40 to 45 percent, from 40 to 50 percent, from 40 to 55 percent, from 45 to 50 percent, from 45 to 55 percent, from 45 to 60 percent, from 50 to 55 percent, from 50 to 60 percent, from 50 to 65 percent, from 55 to 60 percent, from 55 to 65 percent, from 55 to 70 percent, from 60 to 65 percent, from 60 to 70 percent, from 60 to 75 percent, from 65 to 70 percent, from 65 to 75 percent, from 65 to 80 percent, from 70 to 75 percent, from 70 to 80 percent, from 70 to 85 percent, from 75 to 80 percent, from 75 to 85 percent, from 75 to 90 percent, from 80 to 85 percent, from 80 to 90 percent, from 80 to 95 percent, from 85 to 90 percent, from 85 to 95 percent, from 85 to 100 percent, from 90 to 95 percent, from 90 to 100 percent, or from 95 to 100 percent.

In some cases, nucleic acid designed to express one or more transcription factors (or the one or more transcription factors themselves) can be administered to a mammal in need thereof (e.g., a mammal having cancer) to increase the survival rate of the mammal (e.g., increase the five-year relative survival rate of the mammal) by 1, 2, 3, 4, 5, 6, 7, 8, 9, 10, or more years. In some cases, nucleic acid designed to express one or more transcription factors (or the one or more transcription factors themselves) can be administered to a mammal in need thereof (e.g., a mammal having cancer) to increase the survival rate of the mammal (e.g., increase the five-year relative survival rate of the mammal) from 1 to 10 years, such as from 1 to 1.5 years, from 1 to 2 years, from 1 to 2.5 years, from 1.5 to 2 years, from 1.5 to 2.5 years, from 1.5 to 3 years, from 2 to 2.5 years, from 2 to 3 years, from 2 to 3.5 years, from 2.5 to 3 years, from 2.5 to 3.5 years, from 2.5 to 4 years, from 3 to 3.5 years, from 3 to 4 years, from 3 to 4.5 years, from 3.5 to 4 years, from 3.5 to 4.5 years, from 3.5 to 5 years, from 4 to 4.5 years, from 4 to 5 years, from 4 to 5.5 years, from 4.5 to 5 years, from 4.5 to 5.5 years, from 4.5 to 6 years, from 5 to 5.5 years, from 5 to 6 years, from 5 to 6.5 years, from 5.5 to 6 years, from 5.5 to 6.5 years, from 5.5 to 7 years, from 6 to 6.5 years, from 6 to 7 years, from 6 to 7.5 years, from 6.5 to 7 years, from 6.5 to 7.5 years, from 6.5 to 8 years, from 7 to 7.5 years, from 7 to 8 years, from 7 to 8.5 years, from 7.5 to 8 years, from 7.5 to 8.5 years, from 7.5 to 9 years, from 8 to 8.5 years, from 8 to 9 years, from 8 to 9.5 years, from 8.5 to 9 years, from 8.5 to 9.5 years, from 8.5 to 10 years, from 9 to 9.5 years, from 9 to 10 years, or from 9.5 to 10 years.

For example, nucleic acid designed to express one or more neuronal transcription factors (or the one or more neuronal transcription factors themselves) can be administered to a mammal (e.g., a human) having brain cancer as described herein to increase the survival rate of the mammal by, for example, 10, 20, 30, 40, 50, 60, 70, 80, 90, 95, or more percent. In some cases, nucleic acid designed to express one or more neuronal transcription factors (or the one or more neuronal transcription factors themselves) can be administered to a mammal (e.g., a human) having brain cancer as described herein to increase the survival rate of the mammal from 10 to 100 percent, such as from 10 to 15 percent, from 10 to 20 percent, from 10 to 25 percent, from 15 to 20 percent, from 15 to 25 percent, from 15 to 30 percent, from 20 to 25 percent, from 20 to 30 percent, from 20 to 35 percent, from 25 to 30 percent, from 25 to 35 percent, from 25 to 40 percent, from 30 to 35 percent, from 30 to 40 percent, from 35 to 45 percent, from 35 to 50 percent, from 40 to 45 percent, from 40 to 50 percent, from 40 to 55 percent, from 45 to 50 percent, from 45 to 55 percent, from 45 to 60 percent, from 50 to 55 percent, from 50 to 60 percent, from 50 to 65 percent, from 55 to 60 percent, from 55 to 65 percent, from 55 to 70 percent, from 60 to 65 percent, from 60 to 70 percent, from 60 to 75 percent, from 65 to 70 percent, from 65 to 75 percent, from 65 to 80 percent, from 70 to 75 percent, from 70 to 80 percent, from 70 to 85 percent, from 75 to 80 percent, from 75 to 85 percent, from 75 to 90 percent, from 80 to 85 percent, from 80 to 90 percent, from 80 to 95 percent, from 85 to 90 percent, from 85 to 95 percent, from 85 to 100 percent, from 90 to 95 percent, from 90 to 100 percent, or from 95 to 100 percent. In another example, nucleic acid designed to express one or more liver transcription factors (or the one or more liver transcription factors themselves) can be administered to a mammal (e.g., a human) having liver cancer as described herein to increase the survival rate of the mammal by, for example, 10, 20, 30, 40, 50, 60, 70, 80, 90, 95, or more percent. In some cases, nucleic acid designed to express one or more liver transcription factors (or the one or more liver transcription factors themselves) can be administered to a mammal (e.g., a human) having liver cancer as described herein to increase the survival rate of the mammal from 10 to 100 percent, such as from 10 to 15 percent, from 10 to 20 percent, from 10 to 25 percent, from 15 to 20 percent, from 15 to 25 percent, from 15 to 30 percent, from 20 to 25 percent, from 20 to 30 percent, from 20 to 35 percent, from 25 to 30 percent, from 25 to 35 percent, from 25 to 40 percent, from 30 to 35 percent, from 30 to 40 percent, from 35 to 45 percent, from 35 to 50 percent, from 40 to 45 percent, from 40 to 50 percent, from 40 to 55 percent, from 45 to 50 percent, from 45 to 55 percent, from 45 to 60 percent, from 50 to 55 percent, from 50 to 60 percent, from 50 to 65 percent, from 55 to 60 percent, from 55 to 65 percent, from 55 to 70 percent, from 60 to 65 percent, from 60 to 70 percent, from 60 to 75 percent, from 65 to 70 percent, from 65 to 75 percent, from 65 to 80 percent, from 70 to 75 percent, from 70 to 80 percent, from 70 to 85 percent, from 75 to 80 percent, from 75 to 85 percent, from 75 to 90 percent, from 80 to 85 percent, from 80 to 90 percent, from 80 to 95 percent, from 85 to 90 percent, from 85 to 95 percent, from 85 to 100 percent, from 90 to 95 percent, from 90 to 100 percent, or from 95 to 100 percent.

In some cases, nucleic acid designed to express one or more transcription factors (or the one or more transcription factors themselves) can be administered to a mammal in need thereof (e.g., a mammal having cancer) to differentiate cancer cells in the mammal (e.g., to convert cancer cells into terminally differentiated and/or non-dividing cells within the mammal). For example, nucleic acid designed to express one or more neuronal transcription factors (or the one or more neuronal transcription factors themselves) can be administered to a mammal (e.g., a human) having brain cancer (e.g., a glioma such as GBM) as described herein to differentiate, for example, 10, 20, 30, 40, 50, 60, 70, 80, 90, 95, 98, 99, or more percent of the brain cancer cells (e.g., glioma cells) in the mammal into non-cancerous neurons in the brain of the living mammal (e.g., functional neurons that can be integrated into the brain of the living mammal). In some cases, nucleic acid designed to express one or more neuronal transcription factors (or the one or more neuronal transcription factors themselves) can be administered to a mammal (e.g., a human) having brain cancer (e.g., a glioma such as GBM) as described herein to differentiate from 10 to 100 percent, such as from 10 to 15 percent, from 10 to 20 percent, from 10 to 25 percent, from 15 to 20 percent, from 15 to 25 percent, from 15 to 30 percent, from 20 to 25 percent, from 20 to 30 percent, from 20 to 35 percent, from 25 to 30 percent, from 25 to 35 percent, from 25 to 40 percent, from 30 to 35 percent, from 30 to 40 percent, from 35 to 45 percent, from 35 to 50 percent, from 40 to 45 percent, from 40 to 50 percent, from 40 to 55 percent, from 45 to 50 percent, from 45 to 55 percent, from 45 to 60 percent, from 50 to 55 percent, from 50 to 60 percent, from 50 to 65 percent, from 55 to 60 percent, from 55 to 65 percent, from 55 to 70 percent, from 60 to 65 percent, from 60 to 70 percent, from 60 to 75 percent, from 65 to 70 percent, from 65 to 75 percent, from 65 to 80 percent, from 70 to 75 percent, from 70 to 80 percent, from 70 to 85 percent, from 75 to 80 percent, from 75 to 85 percent, from 75 to 90 percent, from 80 to 85 percent, from 80 to 90 percent, from 80 to 95 percent, from 85 to 90 percent, from 85 to 95 percent, from 85 to 100 percent, from 90 to 95 percent, from 90 to 100 percent, or from 95 to 100 percent. In another example, nucleic acid designed to express one or more liver transcription factors (or the one or more liver transcription factors themselves) can be administered to a mammal (e.g., a human) having liver cancer as described herein to differentiate, for example, 10, 20, 30, 40, 50, 60, 70, 80, 90, 95, 98, 99, or more percent of the liver cancer cells in the mammal into non-cancerous hepatocytes in the liver of the living mammal (e.g., functional hepatocytes that can be integrated into the liver of the living mammal). In some cases, nucleic acid designed to express one or more liver transcription factors (or the one or more liver transcription factors themselves) can be administered to a mammal (e.g., a human) having liver cancer as described herein to differentiate from 10 to 100 percent, such as from 10 to 15 percent, from 10 to 20 percent, from 10 to 25 percent, from 15 to 20 percent, from 15 to 25 percent, from 15 to 30 percent, from 20 to 25 percent, from 20 to 30 percent, from 20 to 35 percent, from 25 to 30 percent, from 25 to 35 percent, from 25 to 40 percent, from 30 to 35 percent, from 30 to 40 percent, from 35 to 45 percent, from 35 to 50 percent, from 40 to 45 percent, from 40 to 50 percent, from 40 to 55 percent, from 45 to 50 percent, from 45 to 55 percent, from 45 to 60 percent, from 50 to 55 percent, from 50 to 60 percent, from 50 to 65 percent, from 55 to 60 percent, from 55 to 65 percent, from 55 to 70 percent, from 60 to 65 percent, from 60 to 70 percent, from 60 to 75 percent, from 65 to 70 percent, from 65 to 75 percent, from 65 to 80 percent, from 70 to 75 percent, from 70 to 80 percent, from 70 to 85 percent, from 75 to 80 percent, from 75 to 85 percent, from 75 to 90 percent, from 80 to 85 percent, from 80 to 90 percent, from 80 to 95 percent, from 85 to 90 percent, from 85 to 95 percent, from 85 to 100 percent, from 90 to 95 percent, from 90 to 100 percent, or from 95 to 100 percent of the liver cancer cells in the mammal into non-cancerous hepatocytes in the liver of the living mammal (e.g., functional hepatocytes that can be integrated into the liver of the living mammal).

In some cases, nucleic acid designed to express one or more neuronal transcription factors (or the one or more neuronal transcription factors themselves) can be administered to a mammal in need thereof (e.g., a mammal having brain cancer) to reduce astrogliosis in the mammal. For example, nucleic acid designed to express one or more neuronal transcription factors (or the one or more neuronal transcription factors themselves) can be administered to a mammal (e.g., a human) having brain cancer as described herein to reduce astrogliosis in the mammal by, for example, 10, 20, 30, 40, 50, 60, 70, 80, 90, 95, or more percent. In some cases, nucleic acid designed to express one or more neuronal transcription factors (or the one or more neuronal transcription factors themselves) can be administered to a mammal (e.g., a human) having brain cancer as described herein to reduce astrogliosis in the mammal from 10 to 100 percent, such as from 10 to 15 percent, from 10 to 20 percent, from 10 to 25 percent, from 15 to 20 percent, from 15 to 25 percent, from 15 to 30 percent, from 20 to 25 percent, from 20 to 30 percent, from 20 to 35 percent, from 25 to 30 percent, from 25 to 35 percent, from 25 to 40 percent, from 30 to 35 percent, from 30 to 40 percent, from 35 to 45 percent, from 35 to 50 percent, from 40 to 45 percent, from 40 to 50 percent, from 40 to 55 percent, from 45 to 50 percent, from 45 to 55 percent, from 45 to 60 percent, from 50 to 55 percent, from 50 to 60 percent, from 50 to 65 percent, from 55 to 60 percent, from 55 to 65 percent, from 55 to 70 percent, from 60 to 65 percent, from 60 to 70 percent, from 60 to 75 percent, from 65 to 70 percent, from 65 to 75 percent, from 65 to 80 percent, from 70 to 75 percent, from 70 to 80 percent, from 70 to 85 percent, from 75 to 80 percent, from 75 to 85 percent, from 75 to 90 percent, from 80 to 85 percent, from 80 to 90 percent, from 80 to 95 percent, from 85 to 90 percent, from 85 to 95 percent, from 85 to 100 percent, from 90 to 95 percent, from 90 to 100 percent, or from 95 to 100 percent.

Any appropriate mammal can be treated as described herein. Examples of mammals that can have cancer and can be treated as described herein include, without limitation, humans, non-human primates (e.g., monkeys), dogs, cats, cows, horses, pigs, rats, mice, rabbits, ferrets, and sheep. In some cases, a human having cancer can be treated as described herein to reduce the number of cancer cells within the human, for example, by 10, 20, 30, 40, 50, 60, 70, 80, 90, 95, 98, 99, or more percent. In some cases, a human having cancer can be treated as described herein to reduce the number of cancer cells within the human from 10 to 100 percent, such as from 10 to 15 percent, from 10 to 20 percent, from 10 to 25 percent, from 15 to 20 percent, from 15 to 25 percent, from 15 to 30 percent, from 20 to 25 percent, from 20 to 30 percent, from 20 to 35 percent, from 25 to 30 percent, from 25 to 35 percent, from 25 to 40 percent, from 30 to 35 percent, from 30 to 40 percent, from 35 to 45 percent, from 35 to 50 percent, from 40 to 45 percent, from 40 to 50 percent, from 40 to 55 percent, from 45 to 50 percent, from 45 to 55 percent, from 45 to 60 percent, from 50 to 55 percent, from 50 to 60 percent, from 50 to 65 percent, from 55 to 60 percent, from 55 to 65 percent, from 55 to 70 percent, from 60 to 65 percent, from 60 to 70 percent, from 60 to 75 percent, from 65 to 70 percent, from 65 to 75 percent, from 65 to 80 percent, from 70 to 75 percent, from 70 to 80 percent, from 70 to 85 percent, from 75 to 80 percent, from 75 to 85 percent, from 75 to 90 percent, from 80 to 85 percent, from 80 to 90 percent, from 80 to 95 percent, from 85 to 90 percent, from 85 to 95 percent, from 85 to 100 percent, from 90 to 95 percent, from 90 to 100 percent, or from 95 to 100 percent.

When treating a mammal (e.g., a human) having a cancer as described herein, the cancer can be any type of cancer. As used herein, a mammal refers to any organism classified in the class Mammalia. As used herein, a human refers to the species Homo sapiens. In some cases, a cancer can be a blood cancer. In some cases, a cancer can include one or more solid tumors. In some cases, a cancer can be a luminal cancer. In some cases, a cancer can be a carcinoma cancer. In some cases, a cancer can be a sarcoma cancer. In some cases, a cancer can be a myeloma cancer. In some cases, a cancer can be a leukemia cancer. In some cases, a cancer can be a lymphoma cancer. In some cases, a cancer can be a mixed type cancer. In some cases, a cancer can be a primary cancer. In some cases, a cancer can be a secondary cancer. In some cases, a cancer can be a metastatic cancer. In some cases, a cancer can be stage 0 cancer. In some cases, a cancer can be stage I cancer. In some cases, a cancer can be stage II cancer. In some cases, a cancer can be stage IV cancer. Examples of cancers that can be treated as described herein include, without limitation, brain cancers (e.g., gliomas such as GBM), liver cancers (e.g., HCC), breast cancer, prostate cancer, bone cancer, lung cancer, pancreatic cancer, cervical cancer, uterine cancer, gall bladder cancer, bladder cancer, esophageal cancer, skin cancer, kidney cancer, ovary cancer, and leukemia.

In some cases, the methods described herein can include identifying a mammal (e.g., a human) as having a cancer. Any appropriate method can be used to identify a mammal as having a cancer. For example, imaging techniques, biopsy techniques, cytology techniques, microscopy techniques, histochemical staining techniques, immunohistochemical staining techniques, flow cytometry techniques, image cytometry techniques, and/or genetic testing techniques can be used to identify mammals (e.g., humans) having cancer. In some cases, imaging techniques can be X-ray, computed tomography (CT scan), ultrasound, magnetic resonance imaging (MM), position emission tomography (PET scan), and sonogram. In some cases, biopsy techniques can be fine needle aspiration biopsy, core needle biopsy, vacuum-assisted biopsy, excisional biopsy, shave biopsy, punch biopsy, endoscopic biopsy, laparoscopic biopsy, and, bone marrow aspiration biopsy. In some cases, a cytology technique can be a scrape or a brush cytology technique. In some cases, a microscopy technique can be a light microscopy, an electron microscopy, a laser microscopy, and/or an optical microscopy. In some cases, an histological staining technique can be an hematoxylin and eosin (H&E), an alcian blue stain, an aldehyde fuchsin stain, an alkaline phosphatase stain, a bielschowsky stain, a congo red stain, a crystal violet stain, a fontana-masson stain, a giemsa stain, a luna stain, a nissl stain, a periodic acid schiff stain, a red oil o stain, a reticulin stain, a sudan black b stain, a toluidine blue stain, and/or a van gieson stain. In some cases, a genetic testing technique can be a polymerase chain reaction (PCR), a gene expression microarray technique, RNA sequencing, and/or DNA sequencing.

Once identified as having a cancer of a particular type (e.g., a brain cancer, a liver cancer, a kidney cancer, or a lung cancer), one or more appropriate transcription factors for that cancer cell type can be selected for use as described herein. For example, for brain cancers such as GBM, transcription factors such as NeuroD1, Neurog2, and/or Ascl1 can be selected and used to convert brain cancer cells into non-cancerous cells. For liver cancer cells, transcription factors such as HNF4A, Foxa2, and/or GATA4 can be selected and used to convert liver cancer cells into non-cancerous cells. Other examples of transcription factors that can be selected for particular cancer cell types to convert those particular cancer cells into non-cancerous cells are set forth in Table 1.

TABLE 1
Transcription factors for treating cancer.

| Cancer | |
|---|---|
| cell | Transcription factor(s) (Exemplary GenBank ® Accession |
| type | Nos.) |
| Brain | NeuroD1 (NP_002491 or Q13562.3), NeuroD2, NeuroD4, |
| cell | NeuroD6, Neurog1, Neurog2 (NP_076924.1; EAX06278.1; |
| | or AAH36847.1), Neurog3, Nurr1, Tbr1, Ctip2, Cux1, |
| | Cux2, Dlx2, and/or Ascl1 (NP_004307.2) |
| Liver | HNF4A (XP_005260464.1; NP_000448.3; NP_001274113.1; |
| cell | NP_001274112.1; NP_001274111.1; NP_001245284.1; |
| | NP_001025174.1; NP_787110.2; NP_001025175.1; |
| | NP_849181.1; or NP_849180.1), HNF4B, Foxa1, Foxa2 |
| | (AAH11780.1 or ACA06111.1), GATA1, GATA2, GATA3, |
| | and/or GATA4 (AAI43480.1; NP_001295022.1; |
| | NP_002043.2; NP_001295023.1; or NP_001361203.1) |
| Kidney | HNF-1β (P35680.1; P27889.2; NP_000449.1; |
| cell | NP_001291215.1; or NP_001159395.1), Elf5 (NP_938195.1; |
| | NP_001413.1; NP_001230009.1; or NP_001230010.1), |
| | and/or TFCP2L1 (NP_055368.1; EAW95251.1; EAW95250.1; |
| | or AAH64698.1) |
| Lung | Foxa2/HNF3b (AAH06545.2; NP_068556.2; NP_710141.1; |
| cell | or Q9Y261.1), FoxF1 (NP_001442.2; Q12946.2; EAW95424.1; |
| | or AAH89442.), FoxJ1 (NP_001445.2; or AAH46460.1), |
| | and/or Nkx2-1 (NP_001073136.1; NP_003308.1; |
| | AAH06221.2; or AAH80868.1) |
| Skin | E4F1 (AAH80524.1; or Q66K89.2), Nrf2 (AAB32188.1), |
| cell | Ctip1/BCL11a (NP_001352538.1; Q9H165.2; |
| | NP_001350793.1; or NP_075044.2), and/or Foxn1 |
| | (NP_001356298.1; NP_003584.2; or EAW51092.1) |
| Breast | TP63 (NP_001316893.1; NP_003713.3; NP_001316073.1; |
| cell | NP_001108450.1; or NP_001108451.1), Foxa1 |
| | (NP_004487.2;EAW65844.1; or AAH33890.1), ELF5 |
| | (NP_938195.1; NP_001413.1; NP_001230009.1; or |
| | NP_001230010.1), and/or FoxQ1 (NP_150285.3; |
| | EAW55070.1; or AAH53850.1) |

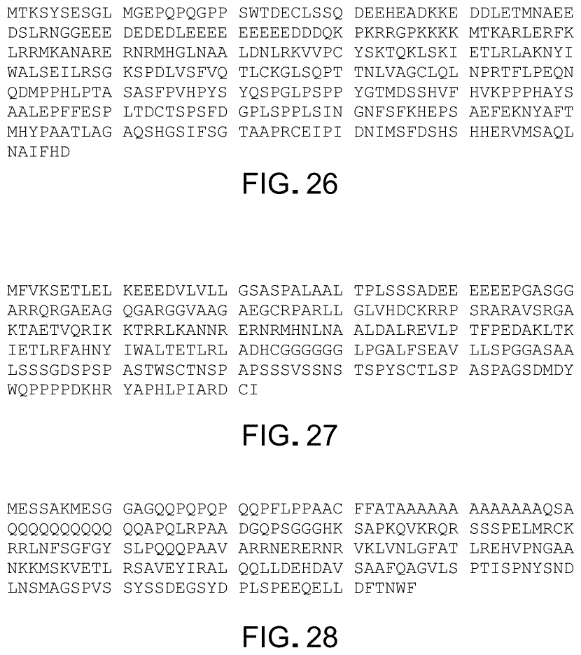

As described herein, a mammal (e.g., a human) having a brain cancer (e.g., a glioma such as GBM) can be treated by administering nucleic acid designed to express one or more neuronal transcription factors within the mammal's brain (e.g., striatum) in a manner that triggers the brain cancer cells (e.g., glioma cells) to form non-cancerous neurons (e.g., functional, near normal, and/or integrated neurons) within the mammal's brain (e.g., striatum). Examples of neuronal transcription factors include, without limitation, NeuroD1 polypeptides, Neurog2 polypeptides, and Ascl1 polypeptides. Examples of NeuroD1 polypeptides include, without limitation, those polypeptides having the amino acid sequence set forth in GenBank® accession number NP_002491 (GI number 121114306) or Q13562.3, or SEQ ID NO:1 (FIG. 26). A NeuroD1 polypeptide can be encoded by a nucleic acid sequence as set forth in GenBank® accession number NM_002500 (GI number 323462174). Examples of Neurog2 polypeptides include, without limitation, those polypeptides having the amino acid sequence set forth in GenBank® accession number NP_076924.1; EAX06278.1; or AAH36847.1, or SEQ ID NO:2 (FIG. 27). A Neurog2 polypeptide can be encoded by a nucleic acid sequence as set forth in GenBank® accession number NM 024019.4. Examples of Ascl1 polypeptides include, without limitation, those polypeptides having the amino acid sequence set forth in GenBank® accession number NP_004307.2 or SEQ ID NO:3 (FIG. 28). An Ascl1 polypeptide can be encoded by a nucleic acid sequence as set forth in GenBank® accession number NM_004316.4.

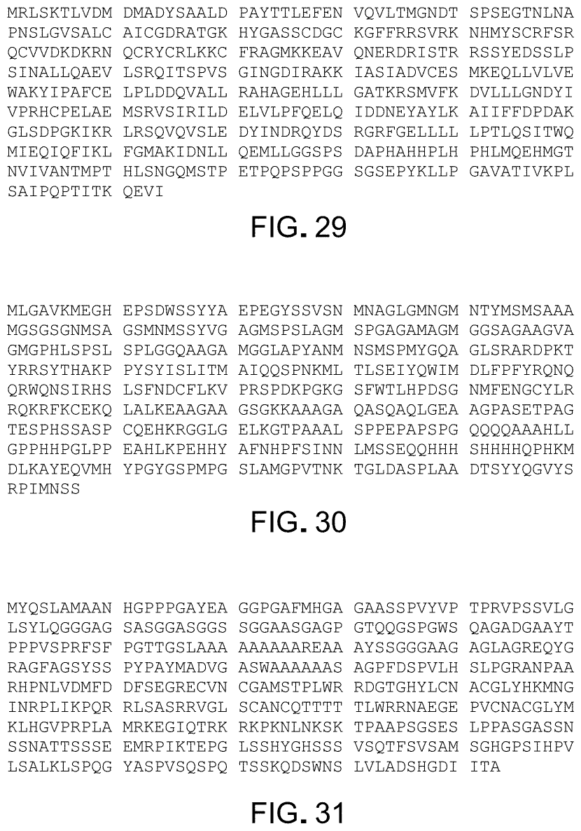

As described herein, a mammal (e.g., a human) having a liver cancer (e.g., HCC) can be treated by administering nucleic acid designed to express one or more liver transcription factors within the mammal's liver in a manner that triggers the liver cancer cells to form non-cancerous hepatocytes (e.g., functional, near-normal, and/or integrated hepatocytes) within the mammal's liver. Examples of liver transcription factors include, without limitation, HNF4A polypeptides, Foxa2 polypeptides, and GATA4 polypeptides. Examples of HNF4A polypeptides include, without limitation, those polypeptides having the amino acid sequence set forth in GenBank® accession number XP_005260464.1; NP_000448.3; NP_001274113.1; NP_001274112.1; NP_001274111.1; NP_001245284.1; NP_001025174.1; NP_787110.2; NP_001025175.1; NP_849181.1; or NP_849180.1, or SEQ ID NO:4 (FIG. 29). A HNF4A polypeptide can be encoded by a nucleic acid sequence as set forth in GenBank® accession number NM_178849.3. Examples of Foxa2 polypeptides include, without limitation, those polypeptides having the amino acid sequence set forth in GenBank® accession number AAH11780.1 or ACA06111.1, or SEQ ID NO:5 (FIG. 30). A Foxa2 polypeptide can be encoded by a nucleic acid sequence as set forth in GenBank® accession number NM 021784.5. Examples of GATA4 polypeptides include, without limitation, those polypeptides having the amino acid sequence set forth in GenBank® accession number AAI43480.1; NP_001295022.1; NP_002043.2; NP_001295023.1; or NP_001361203.1, or and SEQ ID NO:6 (FIG. 31). A GATA4 polypeptide can be encoded by a nucleic acid sequence as set forth in GenBank® accession NM 001308093.3.

Any appropriate method can be used to deliver nucleic acid designed to express one or more transcription factors to cells (e.g., cells within a living mammal). For example, nucleic acid encoding a transcription factor can be administered to a mammal using one or more vectors such as viral vectors. In some cases where two or more nucleic acid designed to express a transcription factor are delivered to cells within a living mammal, separate vectors (e.g., one vector for nucleic acid encoding a first transcription factor, and one vector for nucleic acid encoding a second transcription factor) can be used to deliver the nucleic acids to cells. In some cases where two or more nucleic acid designed to express a transcription factor are delivered to cells within a living mammal, a single vector containing both nucleic acid encoding a first transcription factor and nucleic acid encoding a second transcription factor can be used to deliver the nucleic acids to cells.

Vectors for administering nucleic acid (e.g., nucleic acid designed to express one or more transcription factors) to cells (e.g., cells within a living mammal) can be used to administer nucleic to any appropriate cell. In some cases, a vector can be used to administer nucleic acid encoding a transcription factor to a dividing cell. In some cases, a vector can be used to administer nucleic acid encoding a transcription factor to a non-dividing cell. In some cases, a vector can be used to administer nucleic acid encoding a transcription factor to a cancer cell.

In some cases, vectors for administering nucleic acid (e.g., nucleic acid designed to express one or more transcription factors) to cells (e.g., cells within a living mammal) can be used for transient expression of the transcription factor(s).

In some cases, vectors for administering nucleic acid (e.g., nucleic acid designed to express one or more transcription factors) to cells (e.g., cells within a living mammal) can be used for stable expression of the transcription factor(s). In cases where a vector for administering nucleic acid can be used for stable expression of one or more transcription factors, the vector can be engineered to integrate nucleic acid designed to express one or more transcription factors into the genome of a cell. In some cases, when vector is engineered to integrate nucleic acid into the genome of a cell, any appropriate method can be used to integrate that nucleic acid into the genome of a cell. For example, gene therapy techniques can be used to integrate nucleic acid designed to express one or more transcription factors into the genome of a cell.

Vectors for administering nucleic acids (e.g., nucleic acid encoding one or more transcription factors) to cells (e.g., cells within a living mammal) can be prepared using standard materials (e.g., packaging cell lines, helper viruses, and vector constructs). See, for example, Gene Therapy Protocols (Methods in Molecular Medicine), edited by Jeffrey R. Morgan, Humana Press, Totowa, N.J. (2002) and Viral Vectors for Gene Therapy: Methods and Protocols, edited by Curtis A. Machida, Humana Press, Totowa, N.J. (2003). A vector designed to administer nucleic acid encoding one or more transcription factors to cells (e.g., cells within a living mammal can be an appropriate vector including, without limitation, viral vectors such as adenovirus, adeno-associated virus (AAV), retrovirus, lentivirus, vaccinia virus, herpes virus, papilloma virus, oncolytic virus, and non-viral vectors such as nanoparticles that mimic viral vectors. In some cases, nucleic acid encoding one or more transcription factors can be delivered to cells using adeno-associated virus vectors (e.g., an AAV serotype 2 viral vector, an AAV serotype 5 viral vector, an AAV serotype 9 viral vector, or a recombinant AAV serotype viral vector such as an AAV serotype 2/5 viral vector), lentiviral vectors, retroviral vectors, adenoviral vectors, herpes simplex virus vectors, poxvirus vector, oncolytic vector, or non-viral vectors such as nanoparticles that mimic viral vectors. For example, nucleic acid encoding one or more neuronal transcription factors (e.g., nucleic acid encoding a NeuroD1 polypeptide, nucleic acid encoding a Neurog2 polypeptide, and/or nucleic acid encoding an Ascl1 polypeptide) can be delivered to glial cells (e.g., cancerous glial cells) using one or more retroviral vectors. In another example, nucleic acid encoding one or more liver transcription factors (e.g., nucleic acid encoding a HNF4A polypeptide, nucleic acid encoding a Foxa2 polypeptide, and/or nucleic acid encoding a GATA4 polypeptide) can be delivered to hepatocytes using one or more lentiviral vectors.

In addition to nucleic acid encoding one or more transcription factors, a viral vector can contain regulatory elements operably linked to the nucleic acid encoding a transcription factor. Such regulatory elements can include promoter sequences, enhancer sequences, response elements, signal peptides, internal ribosome entry sequences, polyadenylation signals, terminators, or inducible elements that modulate expression (e.g., transcription or translation) of a nucleic acid. The choice of element(s) that may be included in a viral vector depends on several factors, including, without limitation, inducibility, targeting, and the level of expression desired. For example, a promoter can be included in a viral vector to facilitate transcription of a nucleic acid encoding a transcription factor. A promoter can be constitutive or inducible (e.g., in the presence of tetracycline), and can affect the expression of a nucleic acid encoding a polypeptide in a general or tissue-specific manner. Examples of tissue-specific promoters that can be used to drive expression of a neural transcription factor in glial cells (e.g., cancerous glial cells) include, without limitation, GFAP, NG2, Olig2, CAG, EF1a, Aldh1L1, CMV, and ubiquitin promoters. Examples of tissue-specific promoters that can be used to drive expression of a liver transcription factor in hepatocytes include, without limitation, al-antitrypsin, albumin, AFP, CAG, CMV, EF1a, and ubiquitin promoters.

As used herein, “operably linked” refers to positioning of a regulatory element in a vector relative to a nucleic acid in such a way as to permit or facilitate expression of the encoded polypeptide. For example, a viral vector can contain a glial-specific promoter operably linked to a nucleic acid encoding a neural transcription factor such that it drives transcription in glial cells (e.g., cancerous glial cells). For example, a viral vector can contain a liver-specific promoter operably linked to a nucleic acid encoding a liver transcription factor such that it drives transcription in hepatocytes (e.g., cancerous hepatocytes).

Nucleic acid encoding one or more transcription factors can be administered to a mammal using non-viral vectors. Methods of using non-viral vectors for nucleic acid delivery are described elsewhere. See, for example, Gene Therapy Protocols (Methods in Molecular Medicine), edited by Jeffrey R. Morgan, Humana Press, Totowa, N.J. (2002). For example, nucleic acid encoding one or more transcription factors can be administered to a mammal by direct injection of nucleic acid molecules (e.g., plasmids) comprising nucleic acid encoding one or more transcription factors, or by administering nucleic acid molecules complexed with lipids, polymers, or nanospheres. In some cases, a genome editing technique such as CRISPR/Cas9-mediated gene editing can be used to activate endogenous transcription factor expression.

Nucleic acid encoding a transcription factor can be produced by techniques including, without limitation, common molecular cloning, polymerase chain reaction (PCR), chemical nucleic acid synthesis techniques, and combinations of such techniques. For example, PCR or RT-PCR can be used with oligonucleotide primers designed to amplify nucleic acid (e.g., genomic DNA or RNA) encoding a transcription factor.

In some cases, one or more transcription factors can be administered in addition to or in place of nucleic acid designed to express one or more transcription factors. For example, NeuroD1 polypeptides, Neurog2 polypeptides, and/or Ascl1 polypeptides can be administered to a mammal to trigger brain cancer cells (e.g., glioma cells) within the brain to convert into (e.g., to differentiate into) non-cancerous neurons in the brain of the living mammal (e.g., functional neurons that can be integrated into the brain of the living mammal). In another example, HNF4A polypeptides, Foxa2 polypeptides, and/or GATA4 polypeptides can be administered to a mammal to trigger liver cancer cells within the liver into converting into (e.g., to differentiate into) non-cancerous hepatocytes in the liver of the living mammal (e.g., functional hepatocytes that can be integrated into the liver of the living mammal).

As described herein, nucleic acid designed to express one or more transcription factors (or the one or more transcription factors themselves) can be administered to a mammal (e.g., a human) having cancer to treat the mammal. In some cases, nucleic acid designed to express a polypeptide having the amino acid sequence set forth in SEQ ID NO:1, nucleic acid designed to express a polypeptide having the amino acid sequence set forth in SEQ ID NO:2, and nucleic acid designed to express a polypeptide having the amino acid sequence set forth in SEQ ID NO:3 (or a polypeptide having the amino acid sequence set forth in SEQ ID NO:1, a polypeptide having the amino acid sequence set forth in SEQ ID NO:2, and/or a polypeptide having the amino acid sequence set forth in SEQ ID NO:3) can be administered to a mammal (e.g., a human) having brain cancer (e.g., a glioma such as GBM) as described herein to treat the mammal. For example, a single retroviral vector can be designed to express a polypeptide having the amino acid sequence set forth in SEQ ID NO:1, a polypeptide having the amino acid sequence set forth in SEQ ID NO:2, and a polypeptide having the amino acid sequence set forth in SEQ ID NO:3, and that designed viral vector can be administered to a human having brain cancer to treat the mammal.

In some cases, a polypeptide having an amino acid sequence with at least 85% (e.g., 85%, 90%, 93%, 95%, 96%, 97%, 98%, or 99.0%) sequence identity to the amino acid sequence set forth in SEQ ID NO:1 can be used. For example, a polypeptide containing the entire amino acid sequence set forth in SEQ ID NO:1, except that the amino acid sequence contains from one to ten (e.g., ten, one to nine, two to nine, one to eight, two to eight, one to seven, one to six, one to five, one to four, one to three, two, or one) amino acid additions, deletions, substitutions, or combinations thereof, can be used. In some cases, nucleic acid designed to express a polypeptide containing an amino acid sequence with between 90% and 99% sequence identity to the amino acid sequence set forth in SEQ ID NO:1 can be designed and administered to a mammal (e.g., human) having brain cancer (e.g., a glioma such as GBM) to treat the mammal.

In some cases, a polypeptide having an amino acid sequence with at least 85% (e.g., 85%, 90%, 93%, 95%, 96%, 97%, 98%, or 99.0%) sequence identity to the amino acid sequence set forth in SEQ ID NO:2 can be used. For example, a polypeptide containing the entire amino acid sequence set forth in SEQ ID NO:2, except that the amino acid sequence contains from one to ten (e.g., ten, one to nine, two to nine, one to eight, two to eight, one to seven, one to six, one to five, one to four, one to three, two, or one) amino acid additions, deletions, substitutions, or combinations thereof, can be used. In some cases, nucleic acid designed to express a polypeptide containing an amino acid sequence with between 90% and 99% sequence identity to the amino acid sequence set forth in SEQ ID NO:2 can be designed and administered to a mammal (e.g., human) having brain cancer (e.g., a glioma such as GBM) to treat the mammal.

In some cases, a polypeptide having an amino acid sequence with at least 85% (e.g., 85%, 90%, 93%, 95%, 96%, 97%, 98%, or 99.0%) sequence identity to the amino acid sequence set forth in SEQ ID NO:3 can be used. For example, a polypeptide containing the entire amino acid sequence set forth in SEQ ID NO:3, except that the amino acid sequence contains from one to ten (e.g., ten, one to nine, two to nine, one to eight, two to eight, one to seven, one to six, one to five, one to four, one to three, two, or one) amino acid additions, deletions, substitutions, or combinations thereof, can be used. In some cases, nucleic acid designed to express a polypeptide containing an amino acid sequence with between 90% and 99% sequence identity to the amino acid sequence set forth in SEQ ID NO:3 can be designed and administered to a mammal (e.g., human) having brain cancer (e.g., a glioma such as GBM) to treat the mammal.

In another example, nucleic acid designed to express a polypeptide having an amino acid sequence with at least 85% (e.g., 85%, 90%, 93%, 95%, 96%, 97%, 98%, or 99.0%) sequence identity to the amino acid sequence set forth in SEQ ID NO:1, nucleic acid designed to express a polypeptide having an amino acid sequence with at least 85% (e.g., 85%, 90%, 93%, 95%, 96%, 97%, 98%, or 99.0%) sequence identity to the amino acid sequence set forth in SEQ ID NO:2, and nucleic acid designed to express a polypeptide having an amino acid sequence with at least 85% (e.g., 85%, 90%, 93%, 95%, 96%, 97%, 98%, or 99.0%) sequence identity to the amino acid sequence set forth in SEQ ID NO:3 can be designed and administered to a mammal (e.g., human) having brain cancer (e.g., GBM) to treat the mammal.

In some cases, nucleic acid designed to express a polypeptide having the amino acid sequence set forth in SEQ ID NO:4, nucleic acid designed to express a polypeptide having the amino acid sequence set forth in SEQ ID NO:5, and nucleic acid designed to express a polypeptide having the amino acid sequence set forth in SEQ ID NO:6 (or a polypeptide having the amino acid sequence set forth in SEQ ID NO:4, a polypeptide having the amino acid sequence set forth in SEQ ID NO:5, and/or a polypeptide having the amino acid sequence set forth in SEQ ID NO:6) can be administered to a mammal (e.g., a human) having liver cancer (e.g., HCC) as described herein to treat the mammal. For example, a single lentiviral vector can be designed to express a polypeptide having the amino acid sequence set forth in SEQ ID NO:4, a polypeptide having the amino acid sequence set forth in SEQ ID NO:5, and a polypeptide having the amino acid sequence set forth in SEQ ID NO:6, and that designed viral vector can be administered to a human having liver cancer to treat the mammal.

In some cases, a polypeptide having an amino acid sequence with at least 85% (e.g., 85%, 90%, 93%, 95%, 96%, 97%, 98%, or 99.0%) sequence identity to the amino acid sequence set forth in SEQ ID NO:4 can be used. For example, a polypeptide containing the entire amino acid sequence set forth in SEQ ID NO:4, except that the amino acid sequence contains from one to ten (e.g., ten, one to nine, two to nine, one to eight, two to eight, one to seven, one to six, one to five, one to four, one to three, two, or one) amino acid additions, deletions, substitutions, or combinations thereof, can be used. In some cases, nucleic acid designed to express a polypeptide containing an amino acid sequence with between 90% and 99% sequence identity to the amino acid sequence set forth in SEQ ID NO:4 can be designed and administered to a mammal (e.g., human) having liver cancer (e.g., HCC) to treat the mammal.

In some cases, a polypeptide having an amino acid sequence with at least 85% (e.g., 85%, 90%, 93%, 95%, 96%, 97%, 98%, or 99.0%) sequence identity to the amino acid sequence set forth in SEQ ID NO:5 can be used. For example, a polypeptide containing the entire amino acid sequence set forth in SEQ ID NO:5, except that the amino acid sequence contains from one to ten (e.g., ten, one to nine, two to nine, one to eight, two to eight, one to seven, one to six, one to five, one to four, one to three, two, or one) amino acid additions, deletions, substitutions, or combinations thereof, can be used. In some cases, nucleic acid designed to express a polypeptide containing an amino acid sequence with between 90% and 99% sequence identity to the amino acid sequence set forth in SEQ ID NO:5 can be designed and administered to a mammal (e.g., human) having liver cancer (e.g., HCC) to treat the mammal.

In some cases, a polypeptide having an amino acid sequence with at least 85% (e.g., 85%, 90%, 93%, 95%, 96%, 97%, 98%, or 99.0%) sequence identity to the amino acid sequence set forth in SEQ ID NO:6 can be used. For example, a polypeptide containing the entire amino acid sequence set forth in SEQ ID NO:6, except that the amino acid sequence contains from one to ten (e.g., ten, one to nine, two to nine, one to eight, two to eight, one to seven, one to six, one to five, one to four, one to three, two, or one) amino acid additions, deletions, substitutions, or combinations thereof, can be used. In some cases, nucleic acid designed to express a polypeptide containing an amino acid sequence with between 90% and 99% sequence identity to the amino acid sequence set forth in SEQ ID NO:6 can be designed and administered to a mammal (e.g., human) having liver cancer (e.g., HCC) to treat the mammal.

In another example, nucleic acid designed to express a polypeptide having an amino acid sequence with at least 85% (e.g., 85%, 90%, 93%, 95%, 96%, 97%, 98%, or 99.0%) sequence identity to the amino acid sequence set forth in SEQ ID NO:4, nucleic acid designed to express a polypeptide having an amino acid sequence with at least 85% (e.g., 85%, 90%, 93%, 95%, 96%, 97%, 98%, or 99.0%) sequence identity to the amino acid sequence set forth in SEQ ID NO:5, and nucleic acid designed to express a polypeptide having an amino acid sequence with at least 85% (e.g., 85%, 90%, 93%, 95%, 96%, 97%, 98%, or 99.0%) sequence identity to the amino acid sequence set forth in SEQ ID NO:6 can be designed and administered to a mammal (e.g., human) having liver cancer (e.g., HCC) to treat the mammal.

The percent sequence identity between a particular nucleic acid or amino acid sequence and a sequence referenced by a particular sequence identification number (e.g., SEQ ID NO:1 or SEQ ID NO:2) can be determined as follows. First, a nucleic acid or amino acid sequence is compared to the sequence set forth in a particular sequence identification number using the BLAST 2 Sequences (B12seq) program from the stand-alone version of BLASTZ containing BLASTN version 2.0.14 and BLASTP version 2.0.14. This stand-alone version of BLASTZ can be obtained online at world wide web dot “fr” dot “com/blast” or at world wide web dot “ncbi.nlm.nih” dot “gov”. Instructions explaining how to use the Bl2seq program can be found in the readme file accompanying BLASTZ. Bl2seq performs a comparison between two sequences using either the BLASTN or BLASTP algorithm. BLASTN is used to compare nucleic acid sequences, while BLASTP is used to compare amino acid sequences. To compare two nucleic acid sequences, the options are set as follows: −i is set to a file containing the first nucleic acid sequence to be compared (e.g., C:\seq1.txt); −j is set to a file containing the second nucleic acid sequence to be compared (e.g., C:\seq2.txt); −p is set to blastn; −o is set to any desired file name (e.g., C:\output.txt); −q is set to −1; −r is set to 2; and all other options are left at their default setting. For example, the following command can be used to generate an output file containing a comparison between two sequences: C:\B12seq −i c:\seq1.txt −j c:\seq2.txt −p blastn −o c:\output.txt −q −1 −r 2. To compare two amino acid sequences, the options of Bl2seq are set as follows: −i is set to a file containing the first amino acid sequence to be compared (e.g., C:\seq1.txt); −j is set to a file containing the second amino acid sequence to be compared (e.g., C:\seq2.txt); −p is set to blastp; −o is set to any desired file name (e.g., C:\output.txt); and all other options are left at their default setting. For example, the following command can be used to generate an output file containing a comparison between two amino acid sequences: C:\B12seq c:\seq1.txt −j c:\seq2.txt −p blastp −o c:\output.txt. If the two compared sequences share homology, then the designated output file will present those regions of homology as aligned sequences. If the two compared sequences do not share homology, then the designated output file will not present aligned sequences.

Once aligned, the number of matches is determined by counting the number of positions where an identical nucleotide or amino acid residue is presented in both sequences. The percent sequence identity is determined by dividing the number of matches by the length of the sequence set forth in the identified sequence (e.g., SEQ ID NO:1), followed by multiplying the resulting value by 100. For example, an amino acid sequence that has 340 matches when aligned with the sequence set forth in SEQ ID NO:1 is 95.5 percent identical to the sequence set forth in SEQ ID NO:1 (i.e., 340±356×100=95.5056). It is noted that the percent sequence identity value is rounded to the nearest tenth. For example, 75.11, 75.12, 75.13, and 75.14 is rounded down to 75.1, while 75.15, 75.16, 75.17, 75.18, and 75.19 is rounded up to 75.2. It also is noted that the length value will always be an integer.

When converting a brain cancer cell (e.g., a glioma cell) to a non-cancerous neuron within the brain of a living mammal (e.g., a human) with a brain cancer as described herein (e.g., by administering nucleic acid encoding one or more neuronal transcription factors such as NeuroD1, Neurog2, and/or Ascl1, or one or more neuronal transcription factors themselves), the converted neuron can be any appropriate type of neuron. In some cases, a converted neuron can be DARPP32-positive. In some cases, a converted neuron can be a FoxG1-positive forebrain neuron. In some cases, a converted neuron can be a functional neuron (e.g., can have functional synaptic networks). For example, a functional neuron can be a glutamatergic neuron or a GABAergic neuron. In some cases, a converted neuron can have active electrophysiological properties. In some cases, a converted neuron can be integrated into the brain of a living mammal (e.g., can include axonal projections that extend out of the striatum). In some cases, a converted neuron can exhibit downregulated signaling pathways related to cancer progression (e.g., as compared to the brain cancer cells prior to conversion).

When converting a liver cancer cell to a non-cancerous hepatocyte within the liver of a living mammal (e.g., a human) with a liver cancer as described herein (e.g., by administering nucleic acid encoding one or more liver transcription factors (e.g., nucleic acid encoding HNF4A, Foxa2, and/or GATA4, or one or more liver transcription factors themselves), the converted hepatocyte can be any appropriate type of hepatocyte. In some cases, a converted hepatocyte can be a functional hepatocyte (e.g., can produce cholesterol, bile acids, and/or one or more liver enzymes such as albumin). In some cases, a converted hepatocyte can be integrated into the liver of a living mammal (e.g., can form tight junctions and/or adherins junctions with hepatocytes in the liver of a living mammal). In some cases, a converted hepatocyte can have decreased proliferation (e.g., as compared to the liver cancer cells prior to conversion). In some cases, decreased proliferation can be 10, 20, 30, 40, 50, 60, 70, 80, 90, 95, or more percent. In some cases, decreased proliferation can be from 10 to 100 percent, such as from 10 to 15 percent, from 10 to 20 percent, from 10 to 25 percent, from 15 to 20 percent, from 15 to 25 percent, from 15 to 30 percent, from 20 to 25 percent, from 20 to 30 percent, from 20 to 35 percent, from 25 to 30 percent, from 25 to 35 percent, from 25 to 40 percent, from 30 to 35 percent, from 30 to 40 percent, from 35 to 45 percent, from 35 to 50 percent, from 40 to 45 percent, from 40 to 50 percent, from 40 to 55 percent, from 45 to 50 percent, from 45 to 55 percent, from 45 to 60 percent, from 50 to 55 percent, from 50 to 60 percent, from 50 to 65 percent, from 55 to 60 percent, from 55 to 65 percent, from 55 to 70 percent, from 60 to 65 percent, from 60 to 70 percent, from 60 to 75 percent, from 65 to 70 percent, from 65 to 75 percent, from 65 to 80 percent, from 70 to 75 percent, from 70 to 80 percent, from 70 to 85 percent, from 75 to 80 percent, from 75 to 85 percent, from 75 to 90 percent, from 80 to 85 percent, from 80 to 90 percent, from 80 to 95 percent, from 85 to 90 percent, from 85 to 95 percent, from 85 to 100 percent, from 90 to 95 percent, from 90 to 100 percent, or from 95 to 100 percent In some cases, a converted hepatocyte can have decreased expression of one or more liver cancer markers (e.g., as compared to the liver cancer cells prior to conversion). In some cases, decreased expression of one or more liver cancer markers can be 10, 20, 30, 40, 50, 60, 70, 80, 90, 95, or more percent. In some cases, decreased expression of one or more liver cancer markers can be from 10 to 100 percent, such as from 10 to 15 percent, from 10 to 20 percent, from 10 to 25 percent, from 15 to 20 percent, from 15 to 25 percent, from 15 to 30 percent, from 20 to 25 percent, from 20 to 30 percent, from 20 to 35 percent, from 25 to 30 percent, from 25 to 35 percent, from 25 to 40 percent, from 30 to 35 percent, from 30 to 40 percent, from 35 to 45 percent, from 35 to 50 percent, from 40 to 45 percent, from 40 to 50 percent, from 40 to 55 percent, from 45 to 50 percent, from 45 to 55 percent, from 45 to 60 percent, from 50 to 55 percent, from 50 to 60 percent, from 50 to 65 percent, from 55 to 60 percent, from 55 to 65 percent, from 55 to 70 percent, from 60 to 65 percent, from 60 to 70 percent, from 60 to 75 percent, from 65 to 70 percent, from 65 to 75 percent, from 65 to 80 percent, from 70 to 75 percent, from 70 to 80 percent, from 70 to 85 percent, from 75 to 80 percent, from 75 to 85 percent, from 75 to 90 percent, from 80 to 85 percent, from 80 to 90 percent, from 80 to 95 percent, from 85 to 90 percent, from 85 to 95 percent, from 85 to 100 percent, from 90 to 95 percent, from 90 to 100 percent, or from 95 to 100 percent. An example of a liver cancer marker includes, without limitation, AFP. In some cases, a converted hepatocyte can have increased expression of one or more epithelial-specific markers (e.g., as compared to the liver cancer cells prior to conversion). In some cases, increased expression of one or more epithelial-specific markers can be 10, 20, 30, 40, 50, 60, 70, 80, 90, 95, or more percent. In some cases, increased expression of one or more epithelial-specific markers can be from 10 to 100 percent, such as from 10 to 15 percent, from 10 to 20 percent, from 10 to 25 percent, from 15 to 20 percent, from 15 to 25 percent, from 15 to 30 percent, from 20 to 25 percent, from 20 to 30 percent, from 20 to 35 percent, from 25 to 30 percent, from 25 to 35 percent, from 25 to 40 percent, from 30 to 35 percent, from 30 to 40 percent, from 35 to 45 percent, from 35 to 50 percent, from 40 to 45 percent, from 40 to 50 percent, from 40 to 55 percent, from 45 to 50 percent, from 45 to 55 percent, from 45 to 60 percent, from 50 to 55 percent, from 50 to 60 percent, from 50 to 65 percent, from 55 to 60 percent, from 55 to 65 percent, from 55 to 70 percent, from 60 to 65 percent, from 60 to 70 percent, from 60 to 75 percent, from 65 to 70 percent, from 65 to 75 percent, from 65 to 80 percent, from 70 to 75 percent, from 70 to 80 percent, from 70 to 85 percent, from 75 to 80 percent, from 75 to 85 percent, from 75 to 90 percent, from 80 to 85 percent, from 80 to 90 percent, from 80 to 95 percent, from 85 to 90 percent, from 85 to 95 percent, from 85 to 100 percent, from 90 to 95 percent, from 90 to 100 percent, or from 95 to 100 percent. Examples of epithelial-specific markers include, without limitation, E-cadherin, claudins, and beta-catenin.

Nucleic acid designed to express one or more transcription factors (or the one or more transcription factors themselves) can be administered to a mammal (e.g., a human) having cancer by any appropriate route. In some cases, administration can be local administration. In some cases, administration can be systemic administration. Examples of routes of administration include, without limitation, intravenous, intramuscular, intrathecal, intracerebral, intraparenchymal, subcutaneous, oral, intranasal, inhalation, transdermal, parenteral, intratumoral, retro-ureter, sub-capsular, vaginal, and rectal administration. In cases where multiple rounds of treatment are administered, a first round of treatment can include administering nucleic acid designed to express one or more transcription factors (or the one or more transcription factors themselves) described herein to a mammal (e.g., a human) by a first route (e.g., intravenously), and a second round of treatment can include administering nucleic acid designed to express one or more transcription factors (or the one or more transcription factors themselves) described herein to a mammal (e.g., a human) by a second route (e.g., intratumorally).

In some cases, nucleic acid designed to express one or more transcription factors (or the one or more transcription factors themselves) described herein can be formulated into a composition (e.g., a pharmaceutical composition) for administration to a mammal (e.g., a mammal having, or at risk of having, cancer). For example, nucleic acid designed to express one or more transcription factors (or the one or more transcription factors themselves) can be formulated into a pharmaceutically acceptable composition for administration to a mammal having cancer. In some cases, nucleic acid designed to express one or more transcription factors (or the one or more transcription factors themselves) can be formulated together with one or more pharmaceutically acceptable carriers (additives) and/or diluents. A pharmaceutical composition can be formulated for administration in solid or liquid form including, without limitation, sterile solutions, suspensions, sustained-release formulations, tablets, capsules, pills, powders, wafers, and granules. Pharmaceutically acceptable carriers, fillers, and vehicles that may be used in a pharmaceutical composition described herein include, without limitation, saline (e.g., phosphate-buffered saline, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium tri silicate, polyvinyl pyrrolidone, cellulose-based substances, sodium carboxymethylcellulose, polyacrylates, waxes, polyethylene-polyoxypropylene-block polymers, polyethylene glycol, and wool fat.

In some cases, methods described herein also can include administering to a mammal (e.g., a mammal having cancer) one or more additional agents used to treat a cancer. The one or more additional agents used to treat a cancer can include any appropriate cancer treatment. In some cases, a cancer treatment can include surgery and/or radiation therapy. In some cases, a cancer treatment can include administration of a pharmacotherapy such as a chemotherapy, hormone therapy, targeted therapy, and/or cytotoxic therapy. For example, a mammal having cancer can be administered nucleic acid designed to express one or more transcription factors (or the one or more transcription factors themselves) described herein and administered one or more additional agents used to treat a cancer. In cases where a mammal having cancer is treated with nucleic acid designed to express one or more transcription factors (or the one or more transcription factors themselves) described herein and is treated with one or more additional agents used to treat a cancer, the additional agents used to treat a cancer can be administered at the same time or independently. For example, nucleic acid designed to express one or more transcription factors (or the one or more transcription factors themselves) described herein and one or more additional agents used to treat a cancer can be formulated together to form a single composition. In some cases, nucleic acid designed to express one or more transcription factors (or the one or more transcription factors themselves) described herein can be administered first, and the one or more additional agents used to treat a cancer administered second, or vice versa.

The invention will be further described in the following examples, which do not limit the scope of the invention described in the claims.

### EXAMPLES

### Example 1: Converting Human Glioblastoma Cells into Neurons

GBM is the most prevalent and aggressive adult primary cancer in the central nervous system (CNS). Current standard GBM therapy is surgery, followed by radio- or chemotherapy, but marginal treatment progress has been made due to the heterogeneity and highly invasive nature of GBM.

This Example provides an alternative approach for treating GBM through transcription factor reprogramming (e.g., Neurog2, NeuroD1, and/or Ascl1 reprogramming) of malignant GBM cells into non-proliferative neurons.

### Cell Culture

Human GBM cell lines were purchased from Sigma (U251) or ATCC (U118). U251 cells were cultured in GBM culture medium, which included MEM (GIBCO), 0.2% penicillin/streptomycin (GIBCO), 10% FBS (GIBCO), 1 mM Sodium Pyruvate (GIBCO), 1% Non Essential Amino Acids (NEAA, GIBCO), and 1× GlutMAX (GIBCO). U118 cells were cultured in culture medium including DMEM (GIBCO), 10% FBS, and 1% penicillin/streptomycin.

Human astrocytes were purchased from ScienCell (HA1800, San Diego, USA). Human astrocytes were cultured in human astrocyte medium, which included DMEM/F12 (GIBCO), 10% FBS, 3.5 mM Glucose (Sigma), and 0.2% penicillin/streptomycin, supplemented with B27 (GIBCO), N2 (GIBCO), 10 ng/mL fibroblast growth factor 2 (FGF2, Invitogen), and 10 ng/mL epidermal growth factor (EFG, Invitrogen).

For subculture, cells were trypsinized by 0.25% Trypsin (GIBCO) or TrypLE Select (Invitrogen), centrifuged for 5 minutes at 800 rpm, re-suspended and plated in corresponding culture medium with a split ratio around 1:4. Cells were maintained at 37° C. in humidified air with 5% CO₂.

Reprogramming Human GBM Cells into Neurons

U251 cells were seeded in poly-D-lysine-coated coverslips in 24-well plates at least twelve hours before the virus infection with a density of 10,000 cells per coverslip. GFP, Neurog2, NeuroD1, or Ascl1 retrovirus was added in GBM cells together with 8 μg/mL Polybrene (Santa Cruz Biotechnology). Culture medium was completely replaced by neuronal differentiation medium (NDM) the next day to help with neuronal differentiation and maturation. NDM included DMEM/F12 (GIBCO), 0.4% B27 supplement (GIBCO), 0.8% N₂ supplement (GIBCO), 0.2% penicillin/streptomycin, 0.5% FBS, Vitamin C (5 μg/mL, Selleck Chemicals), Y27632 (1 μM, Tocris), GDNF (10 ng/mL, Invitrogen), BDNF (10 ng/mL, Invitrogen) and NT3 (10 ng/mL, Invitrogen). Cells were maintained at 37° C. in humidified air with 5% CO₂.

Treatment of Human Glioblastoma Cells with Small Molecules

U251 cells were infected by retroviruses expressing Neurog2-GFP or GFP alone; the next day, culture medium was completely replaced by neuronal differentiation medium (NDM) with small molecules, or 0.22% DMSO for control. The infected glioblastoma cells were treated with 5 μM DAPT, 1.5 μM CHIR99021, 5 μM SB431542, 0.25 μM LDN193189, 1 μM SAG, and 1 μM purmorphamine. The small molecule-contained medium was refreshed every 3-4 days. Cells were first treated in small molecules for 12 days and then changed to NDM for desired time periods before immunostaining.

### In Vivo Neuronal Conversion of Human Glioblastoma Cells

In vivo neuronal conversion of human glioblastoma cells was conducted using Rag1 KO immunodeficient mice (B6.12957-Rag1tm1Mom/J, The Jackson Laboratory, Stock #002216). Half a million (5×10⁵) U251 human glioblastoma cells were transplanted into the striatum of Rag1 KO mouse brains using a stereotaxic device (Hamilton). Retroviruses expressing Neurog2-GFP or GFP alone with similar titer were injected intracranially at the same time and location. Mouse brains were harvested and sliced at 1, 2, 4, and 8 weeks post injection. Immunostaining for brain slice sections were the same as cultured cells.

### Data and Statistical Analysis

Cell counting and the fluorescence intensity were performed in a single blind way with randomly chosen fields of random chosen pictures and analyzed by Image J software. Data are represented as mean±SEM. Multiple group comparisons were performed with two-way ANOVA followed with Dunnett's test. Two group comparisons were performed with Student's t test.

### Efficient Neuronal Conversion of Human GBM Cells by Single Neuronal Transcription Factor Neurog2, NeuroD1, or Ascl1

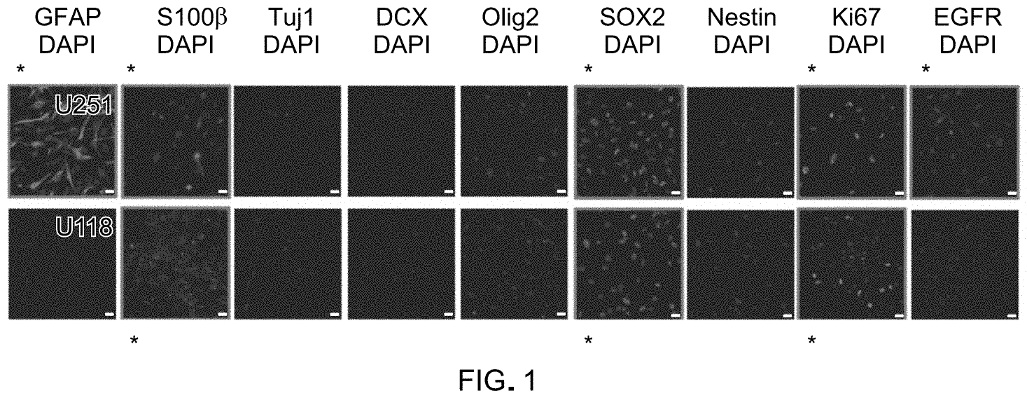

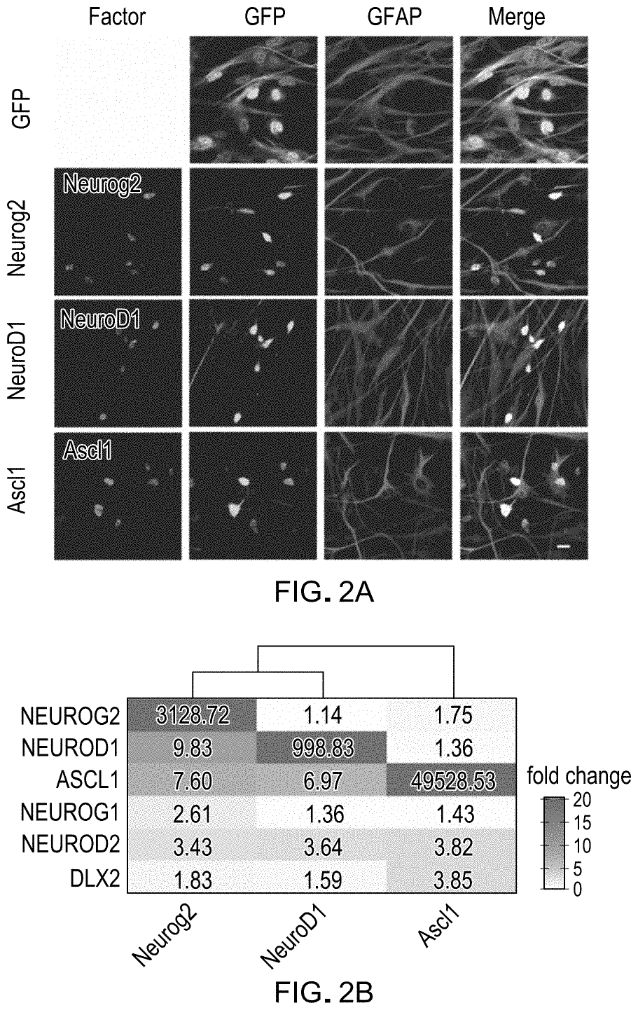

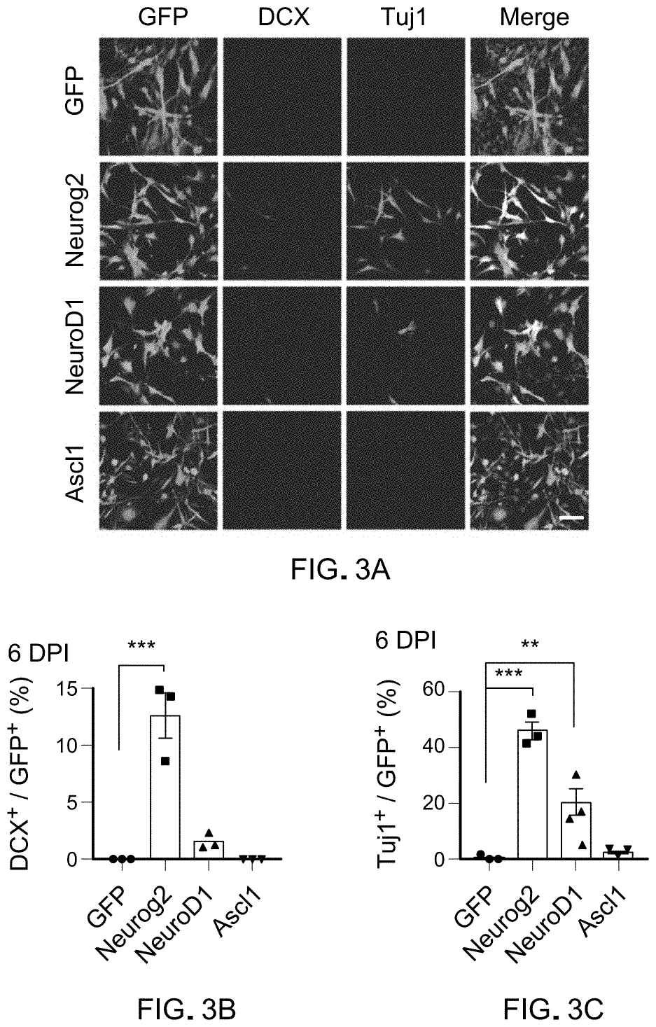

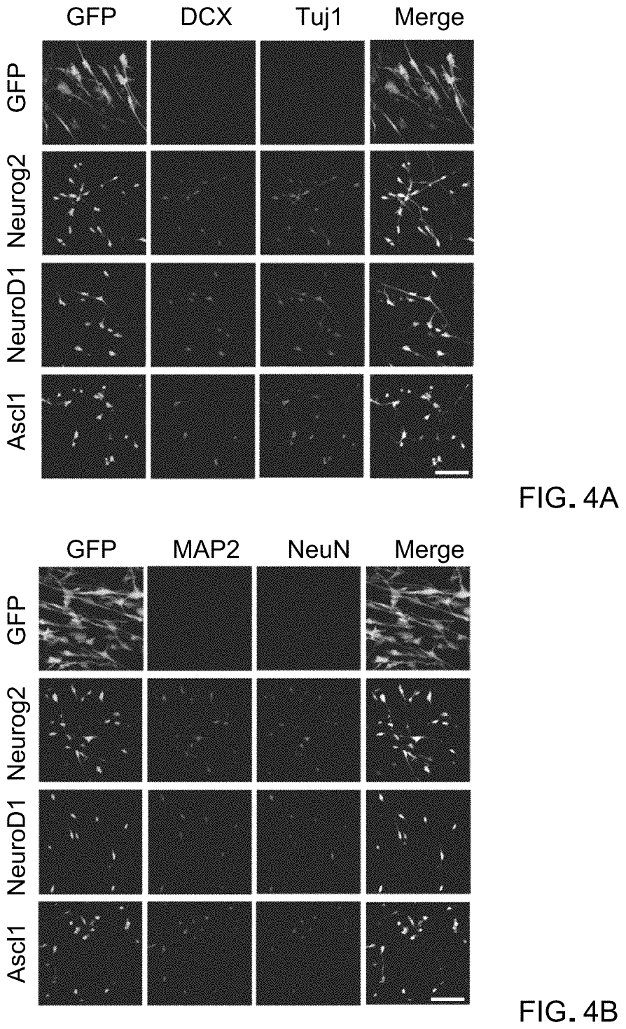

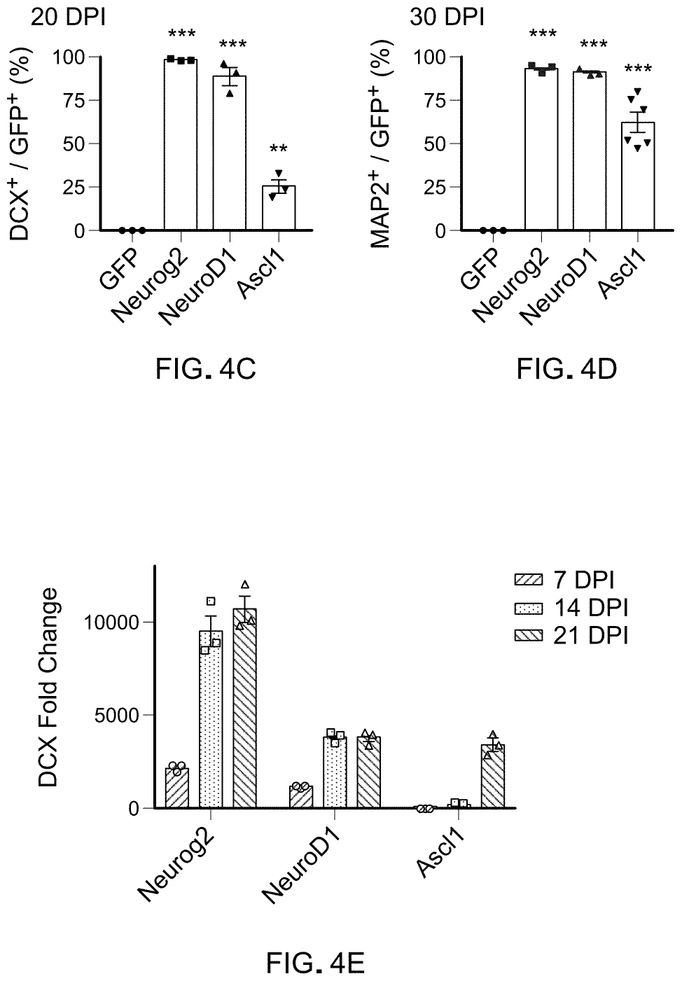

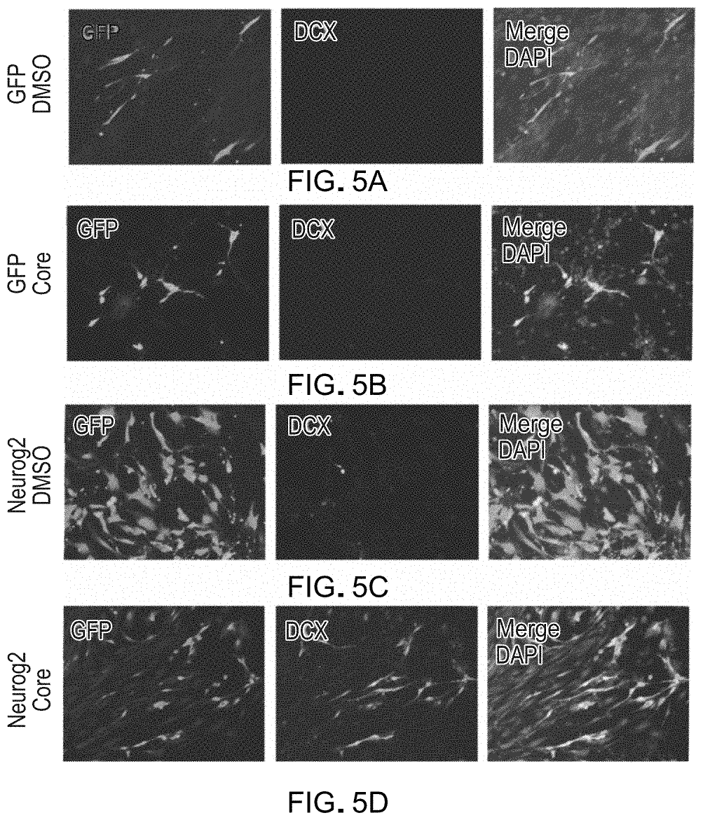

Two different human GBM cell lines (U251, Sigma; U118, ATCC) were used in this study (FIG. 1). To determine if neuronal transcription factors can convert human malignant glioblastoma cells into neurons, transcription factors NeuroD1, Neurog2, and Ascl1 were tested. Given that AAV does not infect cultured glial cells or glioblastoma cells in high efficiency, retroviruses were used to overexpress Neurog2 (CAG::Neurog2-P2A-eGFP), NeuroD1 (CAG::NeuroD1-P2A-eGFP), or Ascl1 (CAG::Ascl1-P2A-eGFP), yielding high infection efficiency in fast-proliferating glioblastoma cells. After twelve-hour incubation with viruses, glioblastoma culture medium was changed to neuronal differentiation medium to help neuronal maturation. Overexpression of Neurog2, NeuroD1, or Ascl1 was confirmed by immunohistochemistry (IHC) (FIG. 2A) and real-time quantitative PCR (RT-qPCR) (FIG. 2B). A few days after transduction, U251 glioblastoma cells began to adopt neuronal morphology after expressing neuronal transcription factors (FIG. 2A), but not the control cells expressing GFP alone (FIG. 2A, top row). A series of pan-neuronal markers were examined for possible neuronal conversion from human glioblastoma cells. Immature neuronal markers DCX and Tuj1 were detected as early as 6 days post viral infection (FIG. 3A-C). By 30 days post infection, mature neuronal makers MAP2 and NeuN were both detected (FIG. 4B). The conversion efficiency was high for all three factors, especially Neurog2 and NeuroD1 (FIG. 4A, quantified in FIG. 4C: Neurog2, 98.2%±0.3%, NeuroD1, 88.7%±5.2%, Ascl1, 24.6%±4.0%, DCX+ neurons/total infected cells at 20 days post infection; FIG. 4B, quantified in FIG. 4D: Neruog2, 93.2%±1.2%, NeuroD1, 91.2%±1.1%, Ascl1, 62.1%±5.9%, MAP2+neurons/total infected cells at 30 days post infection). The neuronal conversion was also confirmed by RT-qPCR, in which the transcriptional activation of DCX was detected after overexpression of Neurog2, NeuroD1, or Ascl1 (FIG. 4E). To test whether neuronal conversion was limited to U251 cells, another human GBM cell line U118 was examined following a similar protocol. It was found that neuronal conversion could be achieved in U118 cells via the combination of neuronal transcription factors and small molecules (e.g., DAPT, CHIR99021, SB431542, and LDN193189) though the conversion efficiency was low (FIG. 5A-D). At 18 days post Neurog2-GFP viral infection with twelve-day small molecule treatment, some U118 cells began expressing neuronal marker DCX (FIG. 5D). However, small molecule treatment alone or Neurog2 alone did not convert U118 cells into neuron-like cells (FIGS. 5B and 5C).

Characterization of the Converted Neurons from Human Glioblastoma Cells

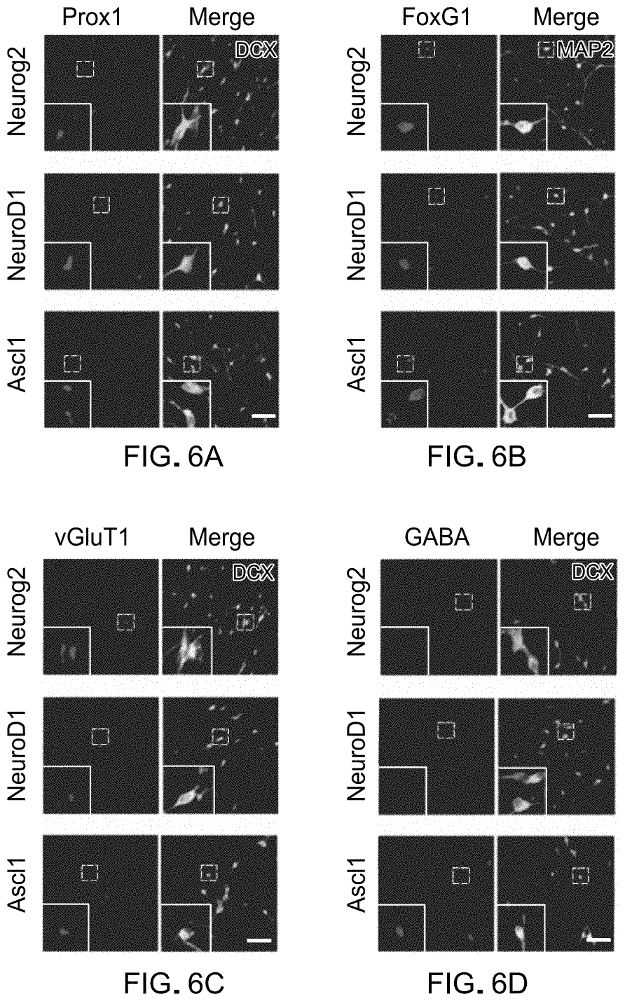

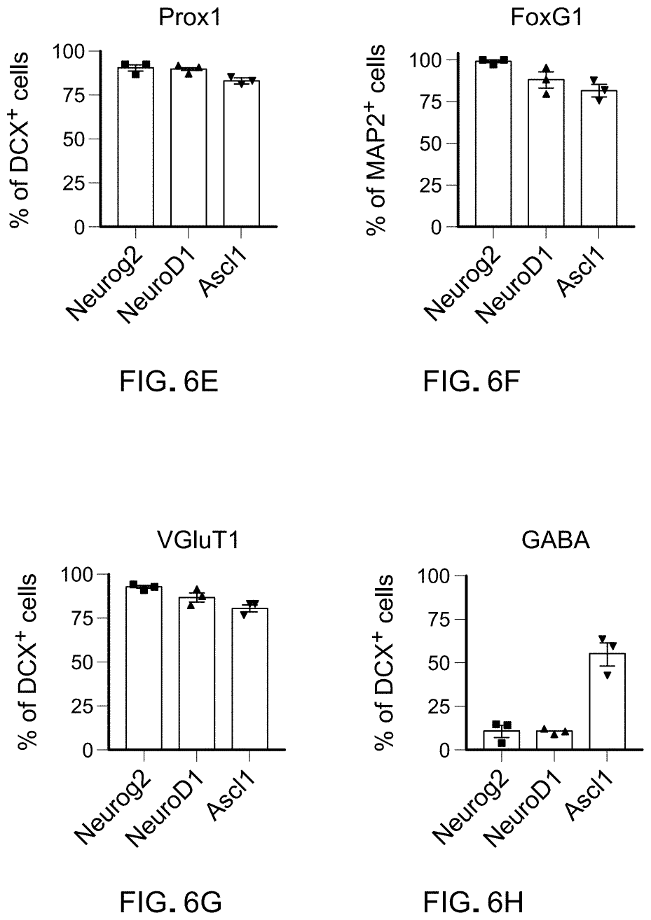

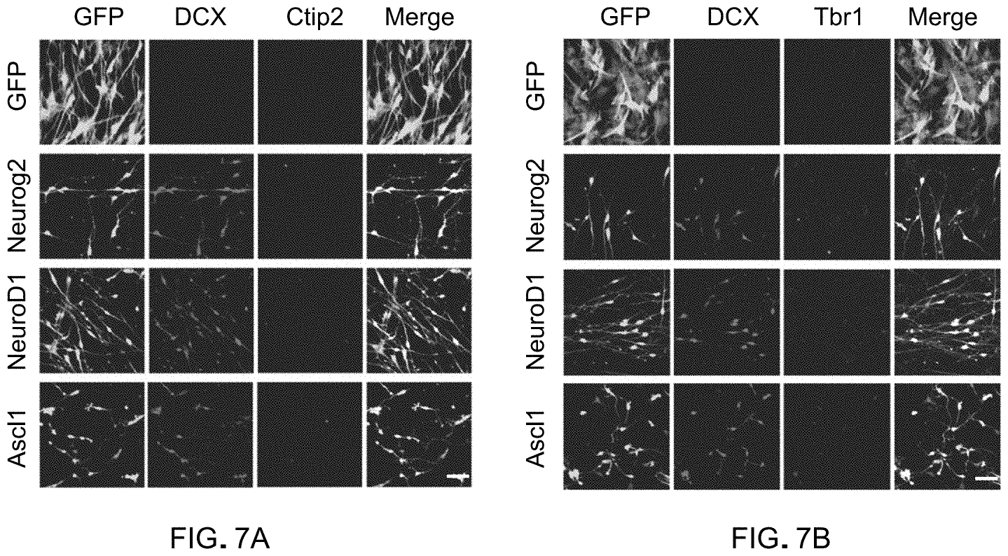

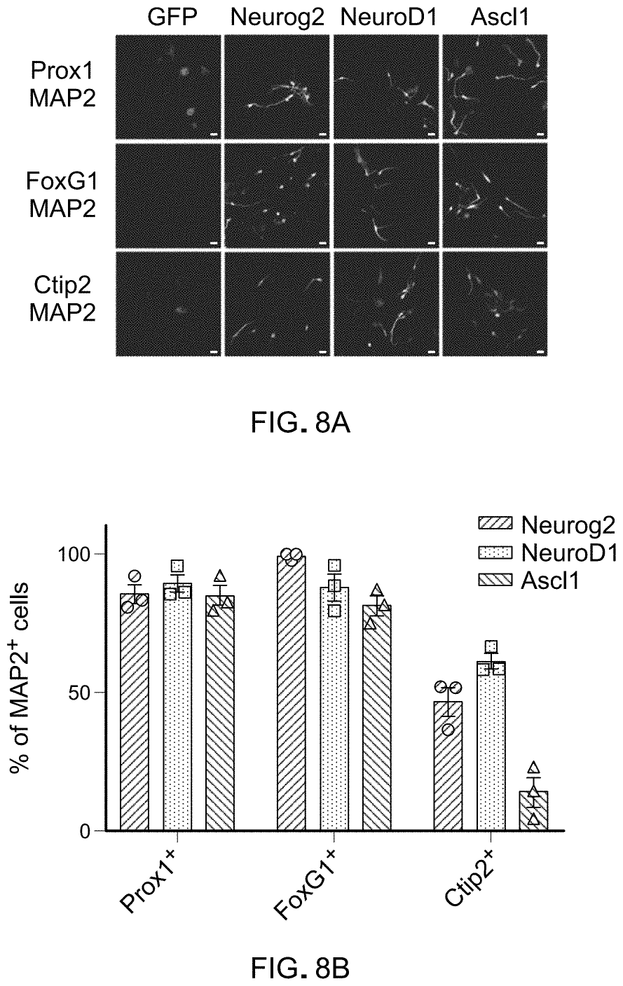

The converted neurons from U251 human glioblastoma cells with neuronal markers expressed in different brain regions was characterized. It was found that a majority of the converted cells was immunopositive for hippocampal granule neuron marker Prox1 (FIG. 6A; quantified in FIG. 6E: Neurog2, 90.4%±1.9%; NeuroD1, 89.9%±1.2%; Ascl1, 83.0%±1.4%; Prox1+/DCX+ cells), and forebrain marker FoxG1 (FIG. 6B; quantified in FIG. 6F: Neurog2, 99.2%±0.8%; NeuroD1, 87.9%±4.8%; Ascl1, 81.3%±3.6%; FoxG1+/MAP2+ cells). Few neurons converted from GBM cells expressed cortical neuron marker Ctip2 or Tbr1 (FIGS. 7A and 7B). These results suggest that the intrinsic imprinting of human glioblastoma cells may be different from astroglial cells and may influence the outcome of cell conversion. Side-by-side comparison was performed with neurons converted from human astrocytes (HA1800, ScienCell, San Diego, USA). A majority of the Neurog2-, NeuroD1-, or Ascl1-converted neurons from human astrocytes was positive for FoxG1 and Prox1, with a significant proportion being immunopositive for Ctip2 (FIGS. 8A and 8B). Therefore, neurons converted from GBM cells shared some common properties with the neurons converted from astrocytes, but differed in the specific neuronal subtypes.

Next, the converted neuronal subtypes were characterized according to the neurotransmitters released, in particular glutamatergic and GABAergic neurons, which are the principal excitatory and inhibitory neurons in the brain, respectively. Most Neurog2-, NeuroD1-, and Ascl1-converted cells were immunopositive for glutamatergic neuron marker VGluT1 (FIG. 6C; quantified in FIG. 6G: Neurog2, 92.8%±0.7%; NeurD1, 86.9%±2.7%; Ascl1, 80.6%±2.1%; VGluT1+/DCX+ cells). The majority of Neurog2- and NeuroD1-converted cells was immunonegative for GABA (FIG. 6D; quantified in FIG. 6H: Neurog2, 11.1%±3.8%; NeuroD1, 8.6%±2.5%; GABA+/DCX+ cells). Roughly half of the Ascl1-converted cells was GABA-positive neurons (FIG. 6D; quantified in FIG. 6H: Ascl1, 49.3%±6.4%, GABA+/DCX+ cells), reflecting the differences among different neuronal conversion factors.

In summary, the majority of the Neurog2-, NeuroD1-, or Ascl1-converted neurons from U251 GBM cells was forebrain glutamatergic neurons, while Ascl1 exhibited a trend for GABAergic neuron generation. These results suggest that the intrinsic GBM cell lineage and the ectopically expressed transcription factors have significant influences on the converted neuronal subtypes.

Fate Change from Glioblastoma Cells to Neurons Induced by Neurog2 Overexpression

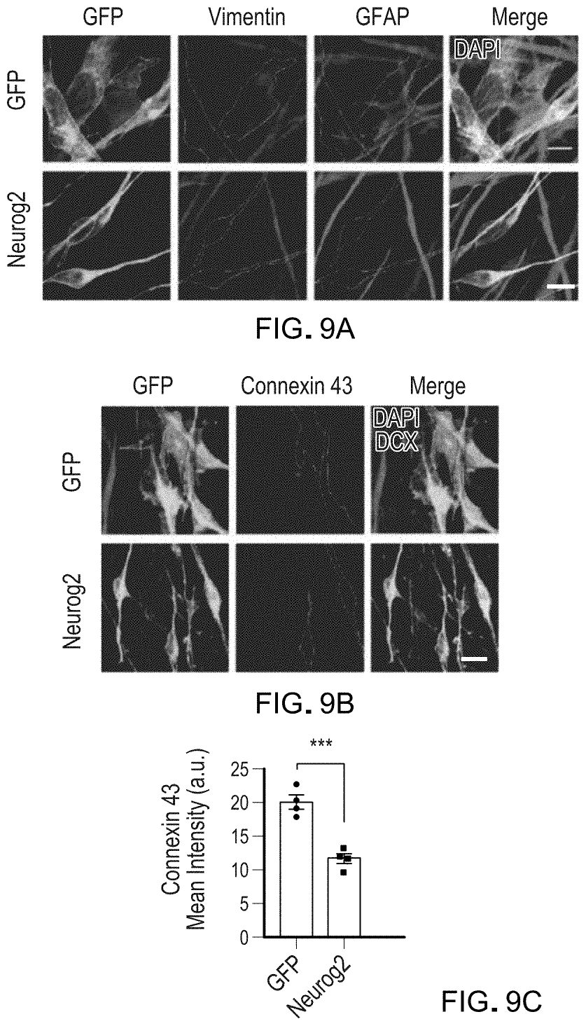

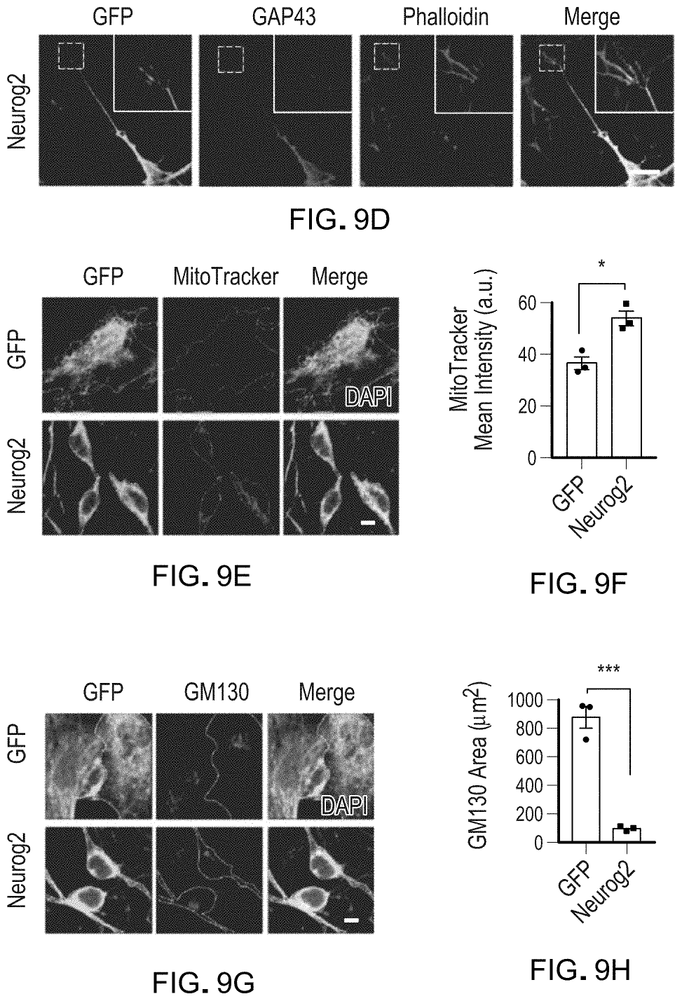

The Neurog2-induced conversion process was investigated. The astrocyte marker GFAP and the epithelial-mesenchymal transition (EMT) marker vimentin were both highly expressed in human U251 cells. After Neurog2 overexpression for 20 days, both GFAP and vimentin were downregulated compared with control (FIG. 9A). This further confirmed the fate change from glioblastoma cells to neurons. In addition, the gap junction marker Connexin 43 was downregulated in U251 glioblastoma cells with Neurog2 overexpression (FIG. 9B; quantified Connexin 43 intensity in FIG. 9C: Neurog2, 19.4±0.7 a.u.; GFP control, 11.6±0.8 a.u.; at 20 days post infection), consistent with the fact that neurons have less gap junctions compared with glial cells. A typical axonal growth cone structure was found in some of the Neurog2-converted neurons (FIG. 9D), with fingerlike filopodia labeled by filamentous actin (F-actin) probe Phalloidin and growth cone marker GAP43 (FIG. 9D).

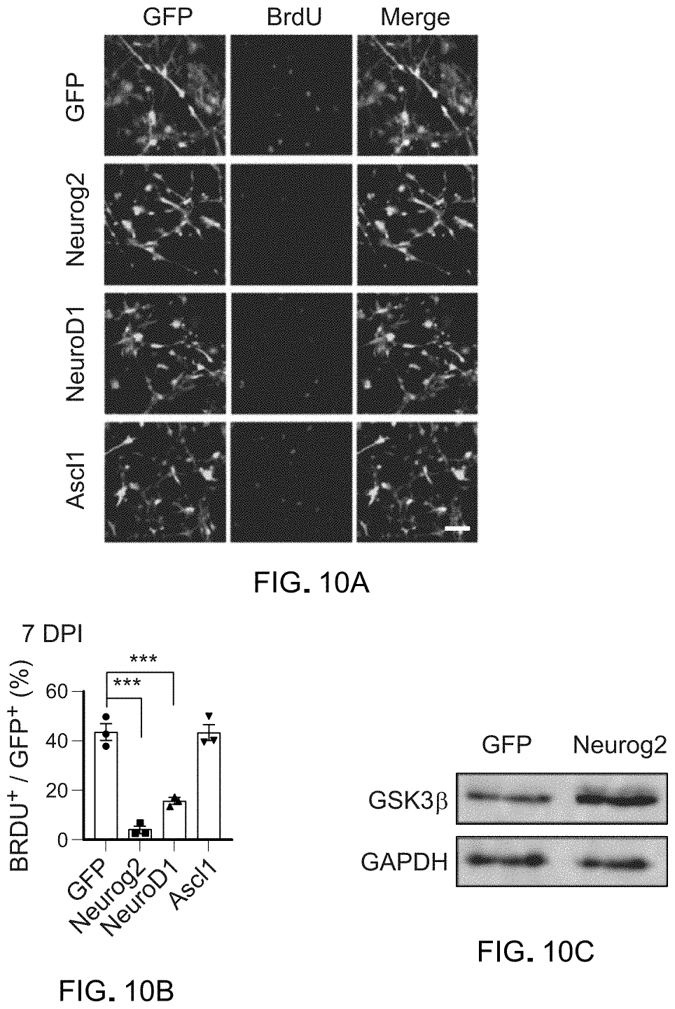

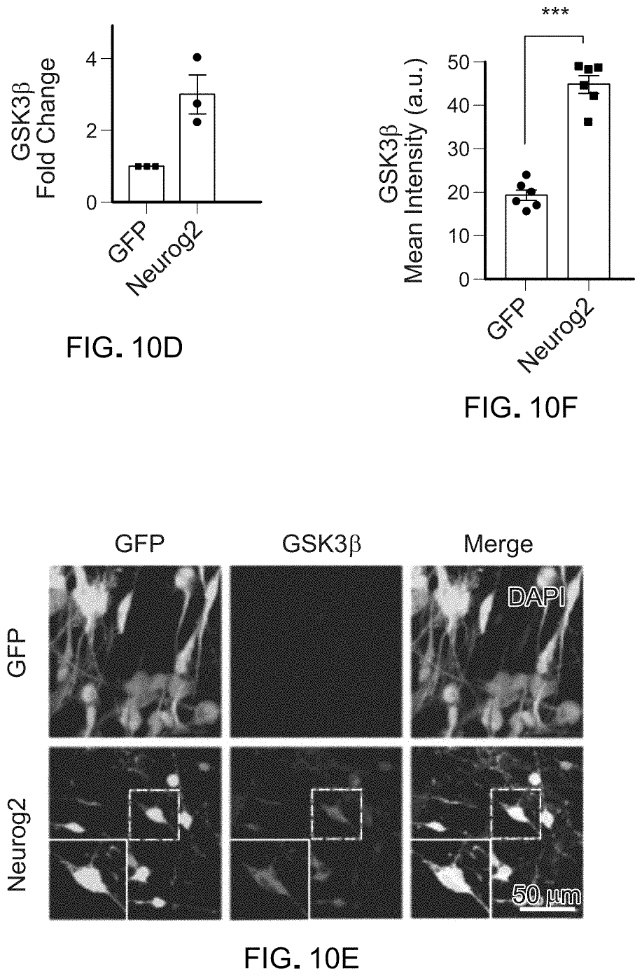

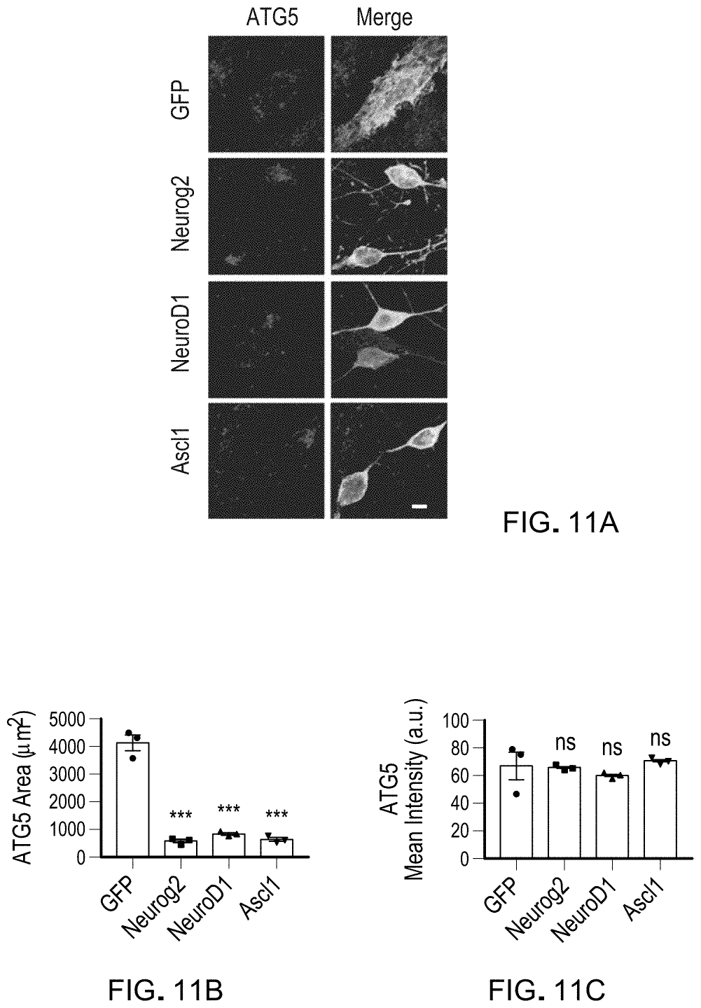

The subcellular changes during Neurog2-induced neuronal conversion of U251 glioblastom cells was investigated. Mitochondria and Golgi apparatus exhibited distinct distribution patterns in Neurog2-converted neurons versus control GBM cells indicated by a Mitotracker labeling assay (FIGS. 10E-F) and Golgi apparatus marker GM130 immunostaining (FIG. 10G-H). Mitochondria are known to locate in areas with a high energy demand. In control U251 cells, mitochondria were distributed in cytoplasm without obvious polarization. In Neurog2-converted neurons, mitochondria were found in both soma and neurites with a polarized distribution pattern in soma (FIG. 10E). In addition, the mean intensity of mitochondria increased in the converted neurons compared with control at 30 days post infection, possibly reflecting both structural and metabolic activity changes (FIG. 10F). The distribution of Golgi apparatus was also distinct between Neurog2-converted neurons and control GBM cells (FIG. 10G). The area of Golgi apparatus was much smaller in Neurog2-converted cells compared with control (FIG. 10H), suggesting possible changes in protein trafficking during the neuronal conversion process. On the other hand, autophagic activity (indicated by immunostaining of an autophagy regulator ATG5) was found to be comparable in the Neurog2-converted cells versus control cells (FIG. 11A-C), suggesting that protein degradation was not significantly affected by the conversion process.

In all, the distinct cellular and subcellular patterns between converted neurons and control glioblastoma cells further demonstrated the fate change from human glioblastoma cells to neurons.

### Functional Analyses of Human Glioblastoma Cell-Converted Neurons

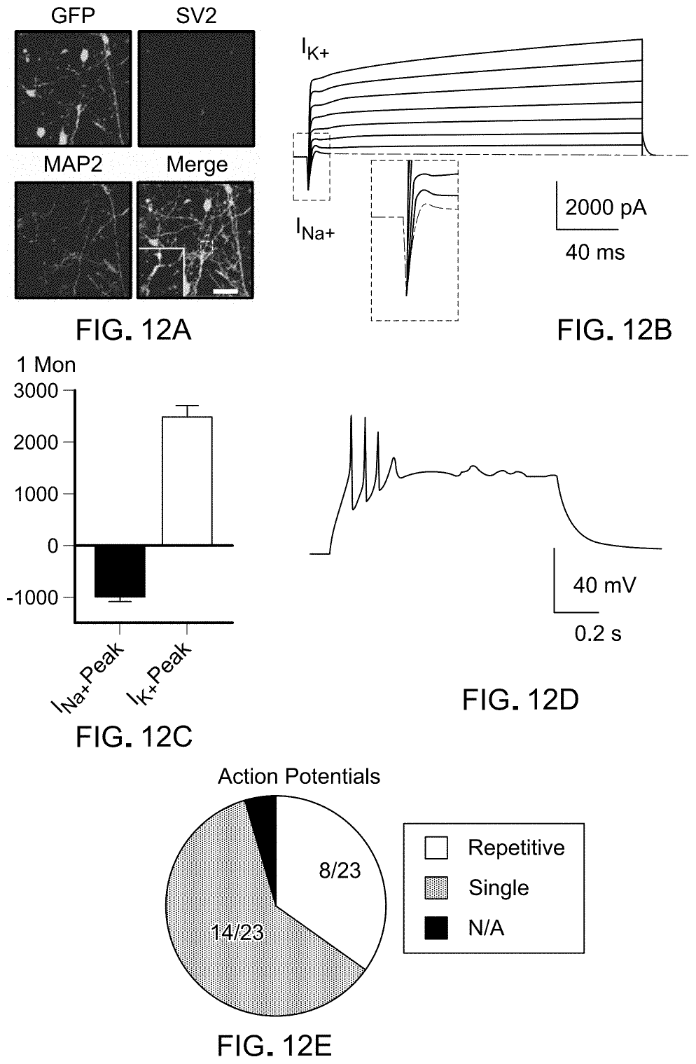

The capability of the Neurog2-converted cells to form synapses was investigated by performing immunostaining for synaptic vesicle marker SV2. Intensive synaptic puncta were detected along MAP2-labeled dendrites in the Neurog2-converted neurons from human GBM cells at 30 days post infection (FIG. 12A). Patch-clamp recordings showed significant sodium and potassium currents in the converted cells at 30 days post infection (FIGS. 12B and 12C). The majority of Neurog2-converted cells fired single action potential (14 out of 23), with a subset of the converted neurons (8 out of 23) firing multiple action potentials (FIGS. 12D and 12E). However, no spontaneous synaptic events were recorded in the Neurog2-converted cells at 30 days post infection, suggesting that the converted neurons may still be immature or perhaps the surrounding glioma cells exert inhibitory effects on synaptic release. In summary, these results indicate that human GBM cells can be reprogrammed into partly functional neuron-like cells by neuronal transcription factors.

### Neuronal Transcription Factors Inhibit GBM Cell Proliferation

Neurons are terminally differentiated non-proliferating cells. Therefore, neuronal transdifferentiation may be a promising strategy to control cancer cell proliferation. Cell proliferation was examined at the early stage of conversion. U251 cells were incubated with 10 mM BrdU for 24 hours to trace the proliferative cells before fixation and staining at 7 days post viral infection (FIG. 10A). Quantification of the percentage of BrdU positive cells showed that the proliferation of Neurog2- and NeuroD1-infected cells decreased significantly compared with GFP control (FIG. 10B: GFP, 64.8%±4.1%; Neurog2, 11.9%±2.9%; NeuroD1, 24.5%±2.4%). The proliferation of Ascl1-converted cells remained active at 7 days post infection (FIG. 10A; quantified in FIG. 10B: Ascl1, 54.6%±1.2%), possibly due to a slow action of Ascl1 in GBM cells (FIG. 4A-D, 3A-C). The proliferation rate of GBM cells was significantly decreased with Neurog2 or NeuroD1 overexpression, consistent with the fast converting speed by Neurog2 and NeuroD1 after infecting GBM cells. These results suggest that in addition to neuronal conversion, ectopic expression of neuronal transcription factors may also be a promising method to control GBM cell proliferation, which is a hallmark of GBM and the major target for GBM treatment.

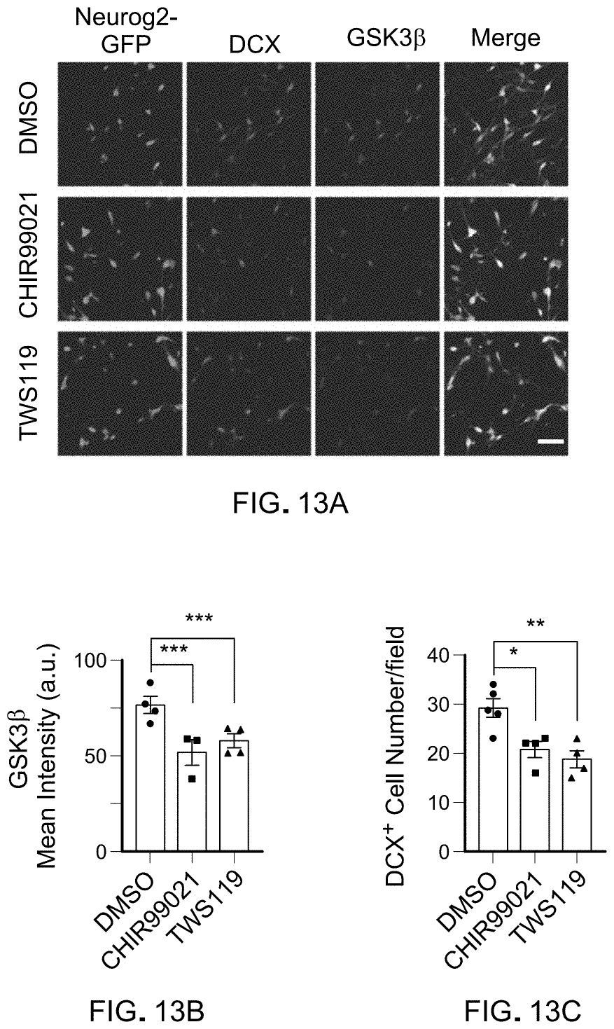

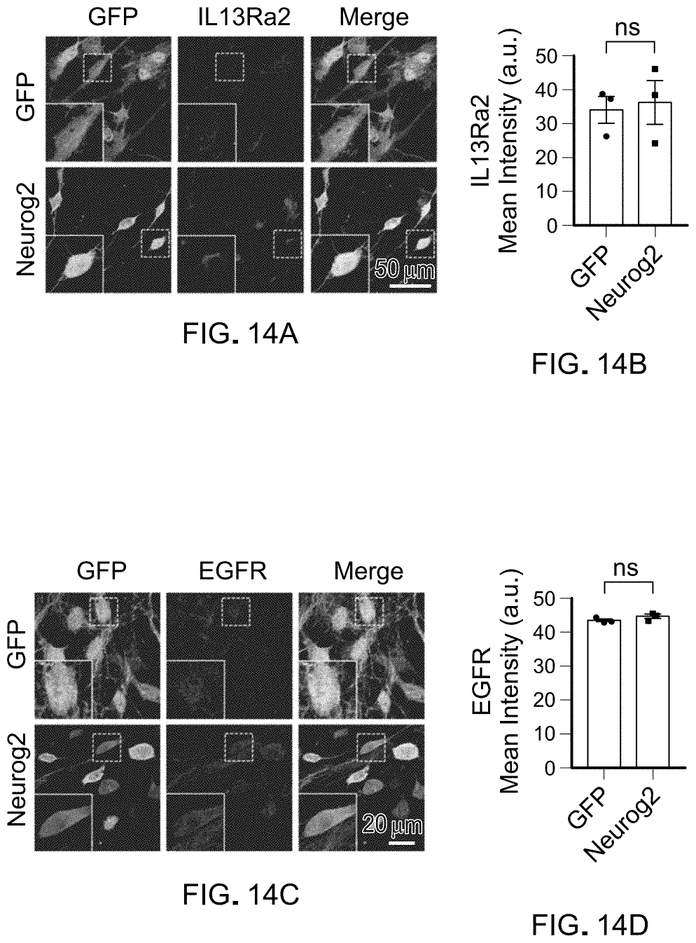

Whether the neuronal conversion would cause any changes of signaling pathways or biomarkers related to glioblastoma progression was also tested. It was found that the expression level of total GSK3β, under western blot analysis, was upregulated at 20 days post Neurog2 virus-infection compared to control U251 cells (FIG. 10C; quantified GSK3β intensity fold change in FIG. 10D: Neurog2, 3.0±0.5). This was further confirmed using immunostaining analysis (FIG. 10E-F). These results demonstrate that GSK3β is involved in neuronal conversion of U251 glioblastoma cells. Neurog2-infected U251 GBM cells were treated with GSK3β antagonists CHIR99021 (5 μM) or TWS119 (10 μM), and it was found that inhibition of GSK3β decreased neuronal conversion efficiency of U251 cells (FIG. 13A-C). In addition, two glioma markers EGFR and IL13Ra2 39-45 were found having steady expression after neuronal conversion at 20 days post Neurog2 infection (FIG. 14A-D), suggesting that the GBM cell-converted neurons may still bear certain imprint of cancer cells, at least for a certain time period after conversion.

### In Vivo Neuronal Conversion of Human Glioblastoma Cells Using a Xenograft Mouse Model

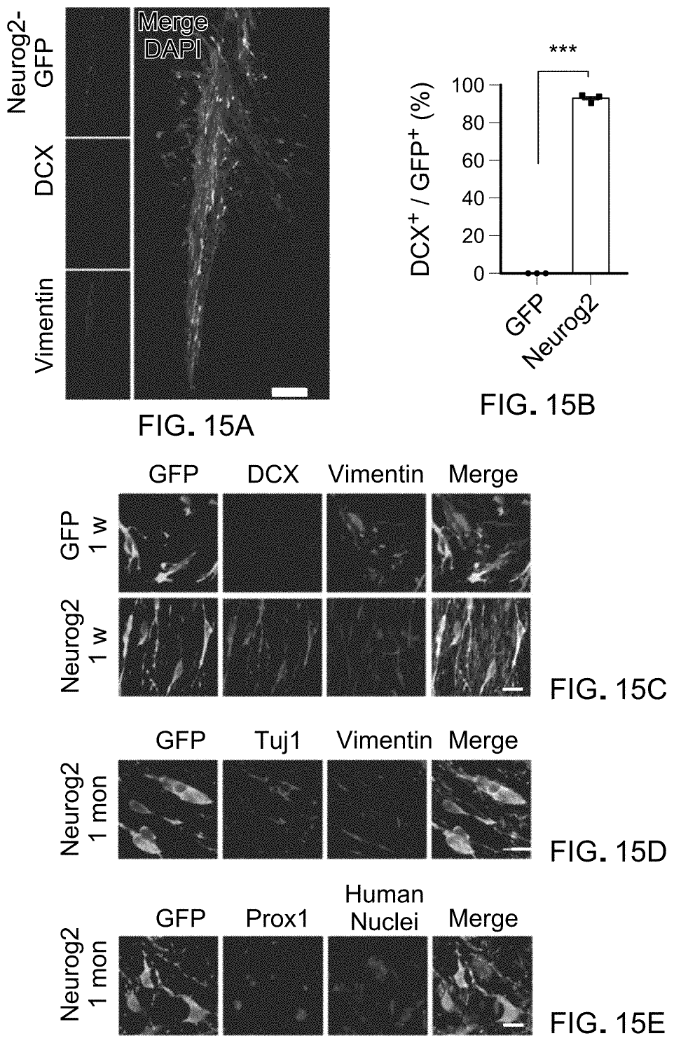

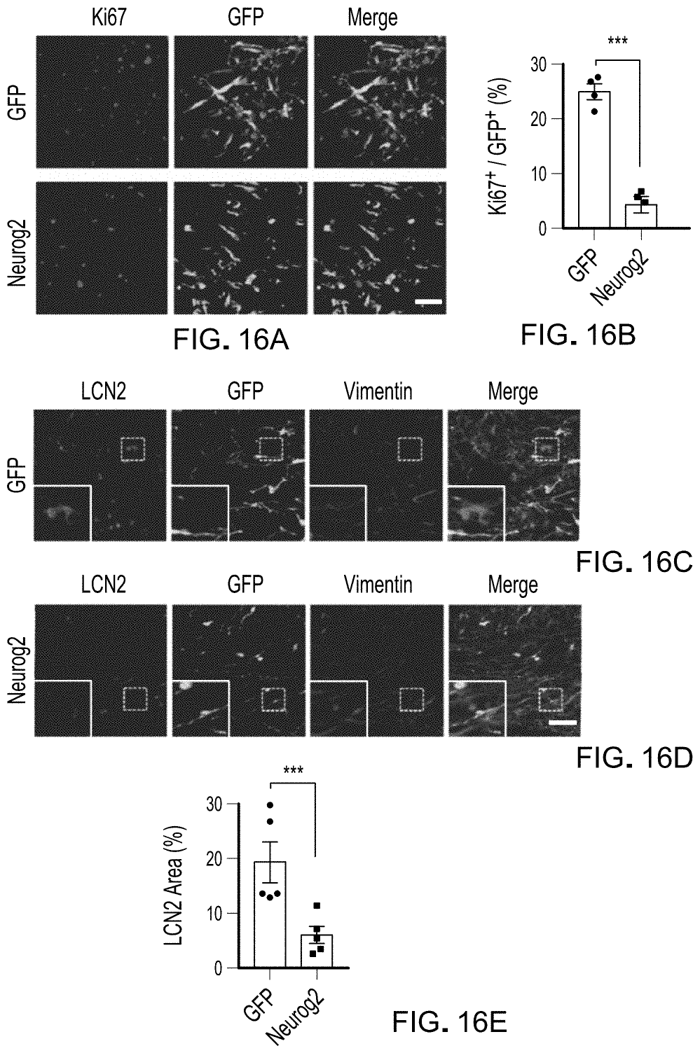

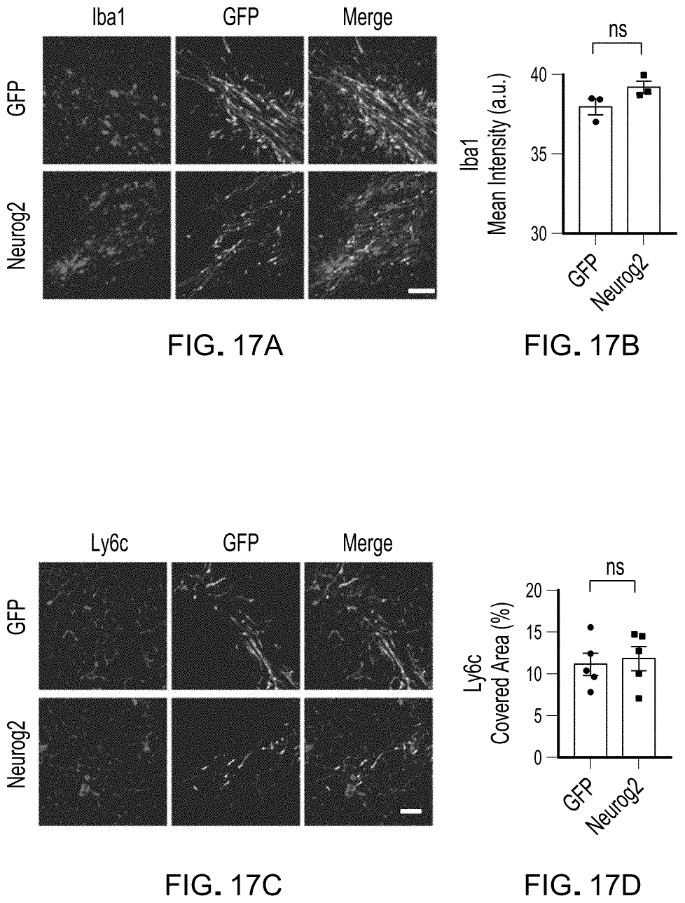

To confirm that the in vitro cell culture results also apply to in vivo environments inside the brain, the conversion capacity of human glioblastoma cells was tested in the mouse brain in vivo. To reduce the complication from immunorejection, intracranial transplantation of human U251 GBM cells (5×10⁵ U251 cells) into the striatum of both sides in Rag1^{-/-} immunodeficient mice was performed (FIG. 15A). Neurog2-GFP or control GFP retroviruses with the same volume (2 μL) and titer (2×10⁵ pfu/mL) were injected in each side of the striatum together with the transplanted GBM cells. Transplanted U251 human GBM cells were identified by vimentin (FIG. 15A) or human nuclei staining (FIG. 15D). Neurog2 overexpression (FIG. 15A) in the transplanted human glioblastoma cells led to an efficient neuronal conversion, indicated by immature neuronal marker DCX (FIGS. 15A-C, quantified in 15B: Neurog2, 92.8%±1.2%, DCX+neurons/total infected cells at 3 weeks post transplantation). Other neuronal makers such as Tuj1 and Prox1 were also detected in the Neurog2-converted cells at one-month post transplantation (FIGS. 15D and 15E). Consistent with conversion results in vitro, the proliferation rate of transplanted glioma cell significantly decreased with Neurog2-GFP viral infection compared with GFP control (FIGS. 16A and 16B). Many LCN2-positive reactive astrocytes were observed in brain areas transplanted with GBM cells, but the number of reactive astrocytes was significantly reduced in the transplantation site with Neurog2 overexpression compared with control side (FIG. 16C-E). Additionally, other possible local environmental factors were also examined in this in vivo model, such as blood vessel or resident microglia distribution. It was found that neither was significantly affected by the Neurog2-induced neuronal conversion (FIG. 17A-D).

In summary, Neurog2, as a representative reprogramming factor, efficiently reprograms human glioblastoma cells into neuron-like cells in vivo in a xenograft mouse model. Moreover, this reprogramming approach significantly inhibits the proliferation of glioma cells and reduces reactive astrogliosis. Together, these results demonstrate that cancer cells (e.g., GBM cells) can be reprogrammed into different subtypes of neurons both in vitro and in vivo, leading to an alternative therapeutic approach to treat cancer (e.g., brain tumors).

### Example 2: Conversion of Liver Cancer Cells into Non-Cancerous Hepatocytes

This Example demonstrates that liver transcription factors (e.g., GATA4, Foxa2, and/or HNF4A) can be used to mediate tumor cell reprogramming and convert tumor cells into normal-like cells, establishing a novel strategy for the treatment of liver cancer or other type of cancers.

### Cell Lines

Human liver cancer cell lines HepG2 and HEK293T (obtained from ATCC) were maintained in DMEM supplemented with 10% FBS and 1% penicillin/streptomycin. All cell lines were routinely treated with MycoSolutions™ (AKRON) to detect Mycoplasma contamination.

### Antibodies

Chicken polyclonal or mouse monoclonal antibodies specific to GFP was purchased from Abcam. Goat polyclonal antibodies against GATA4, Foxa2, and HNF4A proteins were obtained from R&D Systems. Mouse beta-actin monoclonal antibody and goat albumin polyclonal antibody were from Santa Cruz and rabbit GAPDH polyclonal antibody was from Abcam. Rabbit monoclonal antibodies specific to AFP protein and E-cadherin, and mouse monoclonal antibodies specific to HNF4A protein were purchased from Abcam. Rabbit anti-B-catenin polyclonal antibody and goat anti-vimentin polyclonal antibody were obtained from Abcam and R&D Systems, respectively. IRDye 680 Donkey anti-Mouse, IRDye 680 Donkey anti-Rabbit, IRDye 680 Donkey anti-Goat, IRDye 800 Donkey anti-Mouse, IRDye 800 Donkey anti-Rabbit, IRDye 800 Donkey anti-Goat secondary antibodies were purchased from LI-COR.

### Animals

Male immunodeficient athymic nude mice at 4-5 weeks old were obtained from Charles River.

### Generation of Lentiviral Expression Plasmids and Viruses

The AgeI/EcorI fragment of GATA4 (or Foxa2 or HNF4A)—P2A-GFP was cloned into the 3nd generation lentivirus vector, pLJM1 (Addgene), replacing the existing green fluorescent protein (GFP) sequence. The resultant vector plasmids were used to generate the lentiviruses. Lentiviruses were produced by using PEI transfection method. Briefly, 80% confluent of 293T cells grown on 15 cm culture dish were transfected with 12 μg of GATA4 (or Foxa2 or HNF4A)-P2A-GFP encoding lentivirus vector, 2.4 μg of the envelope plasmid pMD2.G (Addgene) encoding VSV glycoprotein G, and 12 μg of the packaging plasmid psPAX2 (Addgene). The virus-containing medium was harvested 72 hours after transfection, filtered to remove cells or cell debris, and concentrated by ultracentrifugation. Viruses titers were determined by infection of HEK293T cells, and GFP positive cells were counted for calculating transducing units per milliliter (TU/mL).

### Cell Growth Assay

Cell growth assays of GATA4, Foxa2, HNF4A, or GFP transduced cells were started with 15,000 cells per 12-well plate. Cells were counted at 6, 24, 48 and 72 hours. At each time point, cells were washed once with phosphate-buffered saline (PBS), and 4% paraformaldehyde (PFA) was added to each well for 15 minutes. Then, cells were stained with 0.1% crystal violet for 20 minutes. The stained crystal violet was extracted by 10% acetic acid and transferred to 96-well plate for the optical density reading at 590 nm by a microplate reader (Bio-Rad Laboratories, CA).

### Mouse Tumor Models

Before transplantation into mice, GATA4, Foxa2, HNF4A, or GFP transduced liver tumor HepG2 cell lines were grown to 80% confluence, counted, and suspended in PBS. Each mouse was subcutaneously injected with 1.0×10⁶ tumor cells into the right flank. Animals were inspected and tumor growth was monitored every 3 to 4 days throughout the experiment. Tumors were measured with a sliding caliper, and tumor volume was calculated using the following formula: 0.5×ab² (a, major axis; b, minor axis). Mice were euthanized, and tumors were dissected and incubated in 4% PFA at 4° C. Tumors were sliced and analyzed by immunofluorescent staining.

### Lentiviral Transduction of Liver Cancer Cells HepG2

For the lentiviral transduction, human liver cancer HepG2 cells were plated in 6 cm culture dish at density of 5×10⁵ cells and incubate for overnight to allow cells attach. HepG2 cells were infected with lentiviruses at an MOI of 1 in 2 mL of fresh DMEM supplemented with 2% FBS. Cultures were incubated at 37° C. for 2 days.

Culture medium was replaced with DMEM containing 10% FBS plus 2 μg/mL of puromycin. Puromycin resistant cells were maintained in DMEM containing 10% FBS plus 2 μg/mL of puromycin.

### Western Blot Analysis.

Culture cells resuspended in 1×PBS were mixed with equal volume of 2× NuPAGE LDS Sample Buffer (Invitrogen). Fresh tumor samples were mixed with 5 volume of 1× RIPA buffer (Invitrogen) and were homogenized for 45 seconds at highest speed by BEAD RUPTOR homogenizer (OMNI International, Inc.). Equal volume of 2× NuPAGE LDS Sample Buffer was added to the lysed tumor samples.

The protein samples were separated on 10% polyacrylamide gels and transferred to polyvinylidene difluoride (PVDF) membranes (Amersham, Piscataway, N.J.). Membranes were blocked in 5% nonfat dry milk and were incubated with primary antibodies, followed with the appropriate secondary antibody. Protein bands detection was carried out using a LI-COR ODYSSEY CLx scanner. Protein bands were quantified by LI-COR Image Studio Ver 3.1 software, and a relative amount of each protein was obtained according to the software instruction.

### Immunofluorescent Staining and Microscopy

Cell cultures grown on coverslips were fixed with 4% paraformaldehyde (PFA) in PBS for 10 minutes. PFA was washed away with PBS, and cells were incubated for 30 minutes in a blocking solution containing 2.5% NDS (Normal Donkey Serum), 2.5% NGS (Normal Goat Serum), and 0.1% Triton X-100 in PBS. Cells were incubated for overnight with primary antibody mixed in blocking solution. Cells were washed three times with PBS and incubated for 1 hour with a mixture of Alexa Fluor 488- or Alexa Fluor 594-secondary antibodies (Jackson ImmunoResearch). Unbinding secondary antibodies were washed away with PBS, and nuclei were stained with DAPI. Cell were visualized with a confocal microscope (Zeiss LSM800).

Tumor sections were permeabilized in PBS with 0.3% Triton X-100 for 1 hour, followed by incubation in blocking solution with 0.3% Triton X-100 in PBS for 1 hour. Tumor sections were incubated with primary antibodies mixed in blocking solution overnight at 4° C. After washing away unbound primary antibodies with PBS, tumor sections were incubated with a mixture of Alexa Fluor 488- or Alexa Fluor 594-secondary antibodies (Jackson ImmunoReaseach) for 1 hour at room temperature, unbound secondary antibodies were washed away with PBS, and nuclei were stained with DAPI. Tumor sections were visualized with a confocal microscope (Zeiss LSM800).

### ELISA Assay

GATA4, Foxa2, HNF4A, or GFP transduced liver tumor HepG2 cell lines were seeded in 12-well plate, incubated for six hours to allow cells attached to plate. The culture medium was replaced with serum free DMEM, and incubation was continued for 16 hours. Culture medium from each cell line was collected, and the albumin amount was measured with the Human Serum Albumin ELISA Kit (Molecular Innovations) according to the kit instructions.

Transduction of Liver Tumor Cell Line HepG2 with Liver Transcription Factors Foxa2, HNF4A, and GATA4

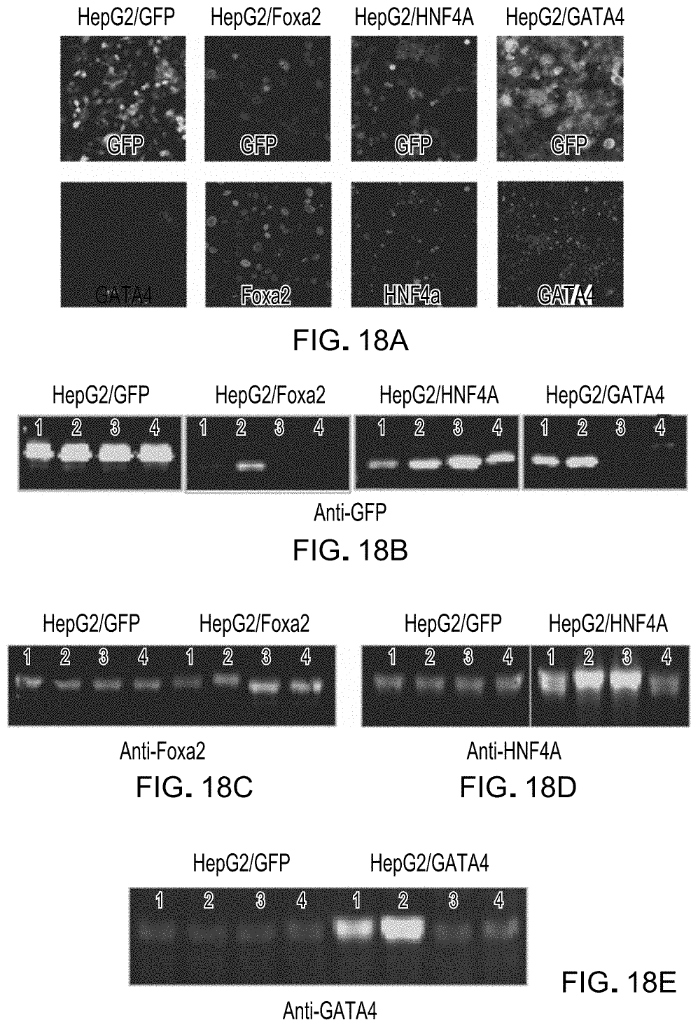

pLJM1 lentiviral vectors carrying Foxa2-P2A-GFP, HNF4A-P2A-GFP, GATA4-P2A-GFP, or GFP were used to infected HepG2 cells. Forty-eight hours later, puromycin was added into the medium to eliminate uninfected cells. Puromycin resistant cells were routinely propagated, and transcription factors or GFP expression in cells were evaluated by immunostaining or western blot. To examine Foxa2, HNF4A, GATA4, and GFP expression and localization, HepG2-Foxa2 (Foxa2-P2A-GFP transduced), HepG2-HNF4A (HNF4A-P2A-GFP transduced), HepG2-GATA4 (GATA4-P2A-GFP transduced), or HepG2-GFP (GFP transduced) cell lines were subjected to fluorescence microscopy. Foxa2, HNF4A, GATA4, and GFP were all highly expressed in each cell line, and transcription factors Foxa2, HNF4A, and GATA4 were localized in the nucleus, while GFP was distributed to the whole cell body (FIG. 18A). Every cell in each transduced line expressed the transduced vector. The expression of transcription factors Foxa2, HNF4A, and GATA4 was further validated by western blot (FIGS. 18B, C, D, and E).

### GATA4 Elevates Endogenous Foxa2 Polypeptide Levels

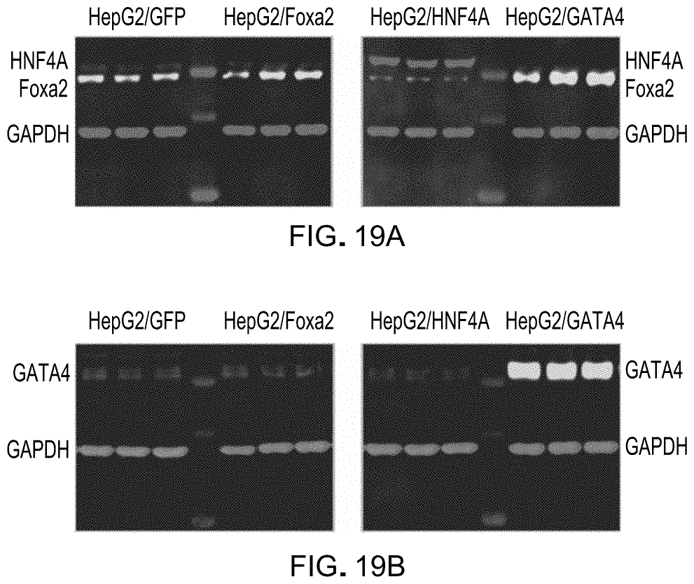

Liver transcription factors Foxa2, HNF4A, and GATA4 were used to transduce HepG2 cells individually. Western blot analysis indicated that GATA4 overexpression increased endogenous Foxa2 expression, and that HNF4A overexpression decreased Foxa2 expression (FIG. 19A). It was observed that both HNF4A and Foxa2 overexpression did not affect endogenous GATA4 expression (FIG. 19B).

### Proliferation in Reprogrammed HepG2 Cells

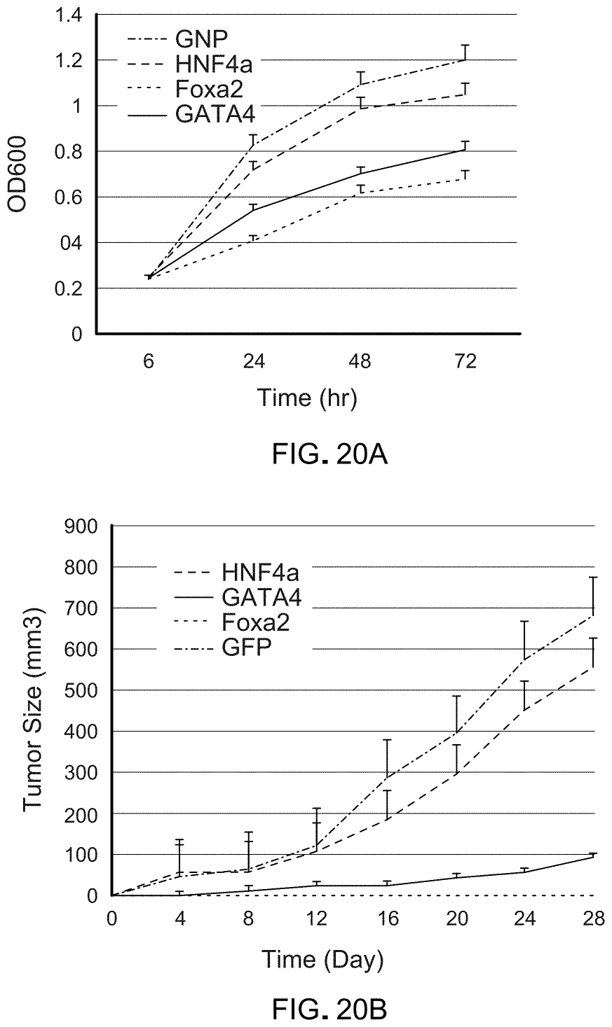

To examine the functional relevance of liver transcription factors expression for liver tumor cell HepG2 growth, GATA4, Foxa2, HNF4A, or GFP transduced cell lines were cultured in 12-well dish. At 6-, 24-, 48-, and 72-hour time points, cells from each cell line were fixed with 4% PFA and stained with 0.1% crystal violet. Stained crystal violet was extracted by 10% acetic acid, and relative growth rates of GATA4, Foxa2, HNF4A, or GFP transduced cell lines were compared by spectrophotometric measurement. As shown in FIG. 20A, cell lines transduced with GATA4, Foxa2, or HNF4A exhibited reduced growth rates compared with the GFP transduced control cell line. Furthermore, Foxa2 and GATA4 mediated cell lines achieved much lower cell growth rate.

To examine in vivo growth rates, GATA4, Foxa2, HNF4A, or GFP transduced cell lines were subcutaneously xenografted into a nude mouse model. Nude mice were randomly assigned into 4 groups with 6 mice/group, and 1×10⁶ cells transduced with GATA4, Foxa2, HNF4A, or GFP were implanted into the flank of nude mice. After 4 days, GFP and HNF4A transduced cell lines started to form tumors. The Foxa2 transduced cell line did not form any visible tumor. The GATA4 transduced cell line showed small tumor growth at later time points. The results revealed that Foxa2 or GATA4 in reprogrammed HepG2 cells decreased cell proliferation (FIG. 20B).

### Function of Reprogrammed HepG2 Cells

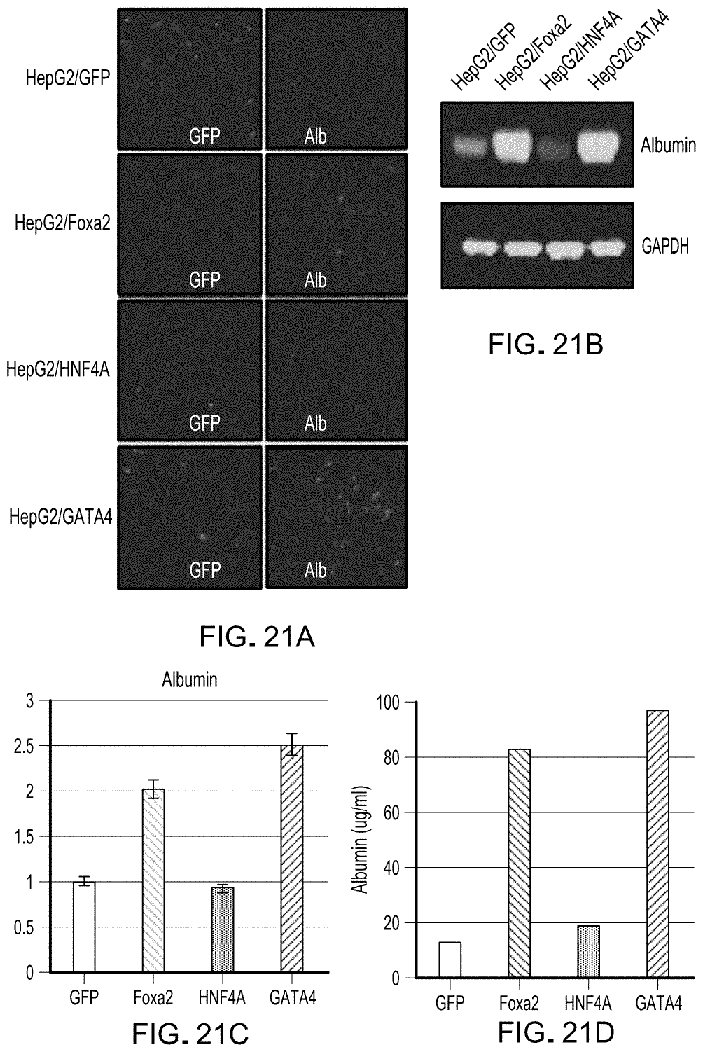

Foxa2, GATA4, HNF4A, or GFP transduced HepG2 cells were stained with anti-albumin antibody to examine albumin production in the lines. As shown in FIG. 21A, both Foxa2 and GATA4 transduced cell lines produced increased albumin as compared with HNF4A and GFP transduced cell lines tested by immunostaining. This result was confirmed by western blot analysis as showed in FIG. 21B. The increased amount of albumin in Foxa2 and GATA4 transduced cell lines was more than 2 fold (FIG. 21C). The albumin in Foxa2 and GATA4 transduced cell lines was able to secret outside of cells. Compared with GFP transduced cell line, the secretion of albumin from Foxa2 or GATA4 transduced cell lines was increased more than 4-fold detected by ELISA (FIG. 21D). Thus, Foxa2 or GATA4 transduction and overexpression promoted the transduced cell lines to exhibit physiological properties of normal liver cells.

### Liver Cancer Markers in Reprogrammed HepG2 Cells

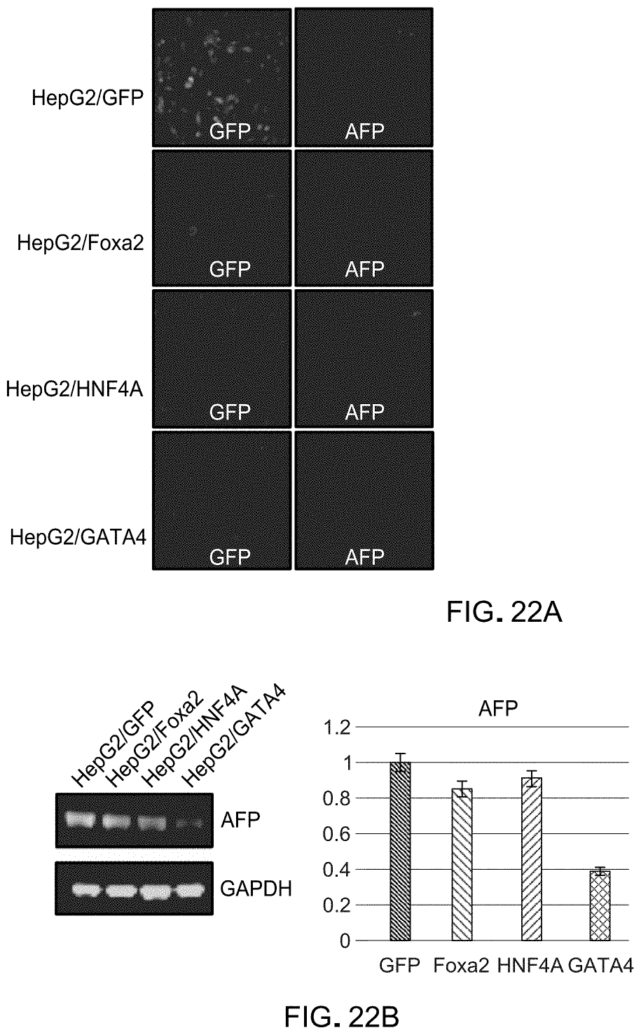

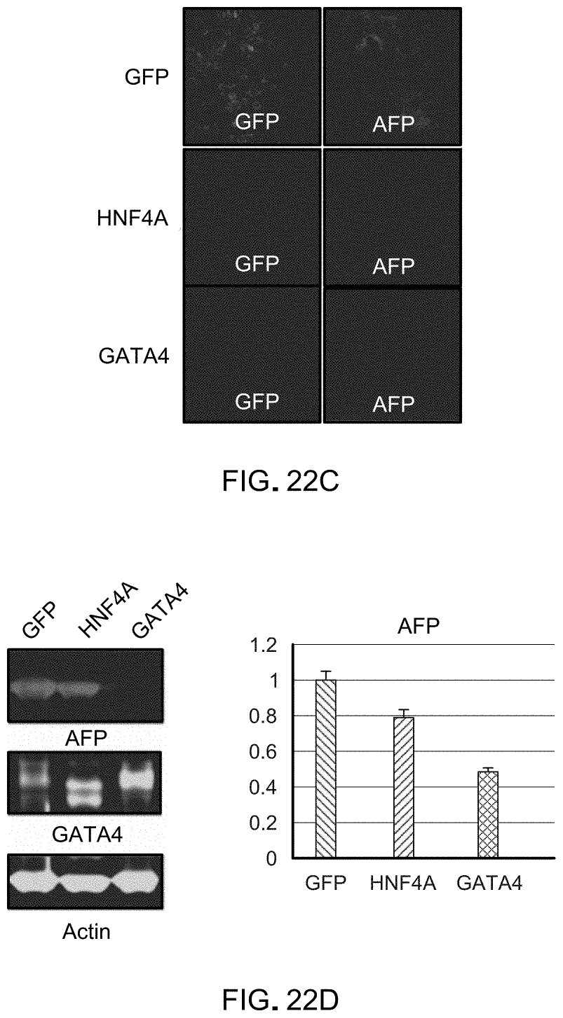

AFP expressed in GATA4, Foxa2, HNF4A, or GFP transduced cell lines localized in the cytosol (FIG. 22A) as shown by immunostaining. In the GATA4 and the Foxa2 transduced cell lines, AFP expression was reduced. By western blot examination, the AFP expression amount in the GATA4 cell line was reduced more 60 percent as compared with GFP transduced cell line (FIG. 22B). For in vivo studies, HepG2 tumors were developed in nude mice with subcutaneously xenografted GATA4, HNF4A, or GFP cell lines, and tumor samples were collected. The Foxa2 transduced cell line lost the ability of tumor formation. Tumors formed with GATA4, HNF4A, or GFP cell lines were fixed in PFA, cut, and analyzed by Immunofluorescence. As shown in FIG. 22C, AFP produced in the GATA4 cell line was decreased, and AFP produced in the HNF4A cell line was mildly reduced. The fresh HepG2 tumor samples developed with GATA4, HNF4A, or GFP cell lines were also subjected to western blot analysis. GATA4 was overexpressed in the tumors formed from the GATA4 cell line, and AFP expression level was reduced (FIG. 22D). Decreased AFP expression was observed both the in vivo and in vitro tests using the GATA4 cell line.

### Liver Cell Markers in Reprogrammed HepG2 Cells

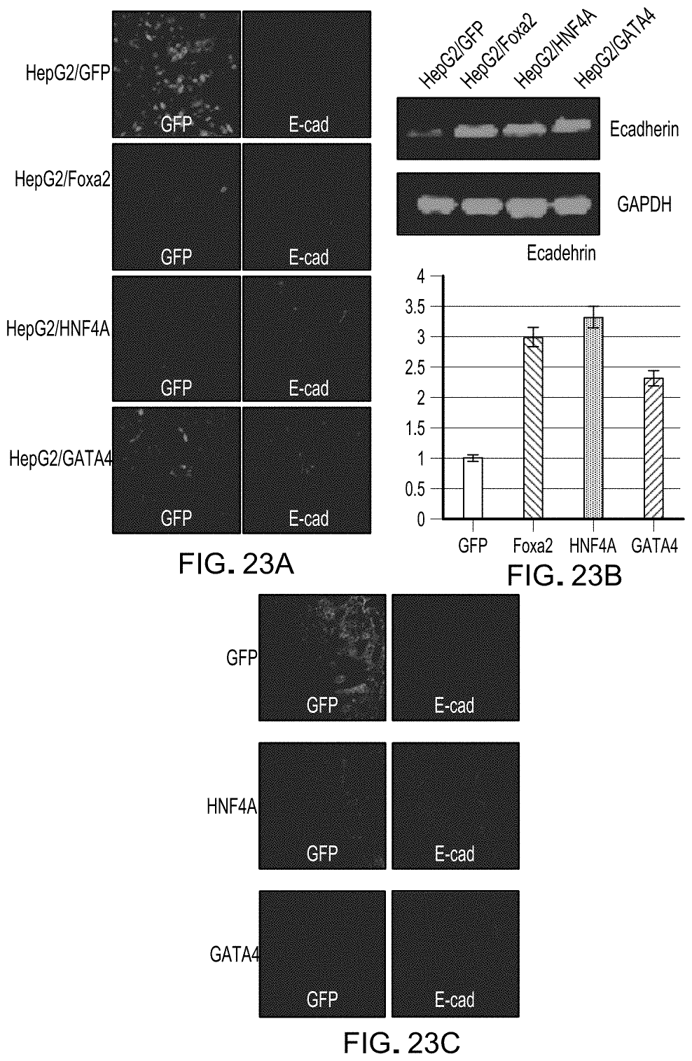

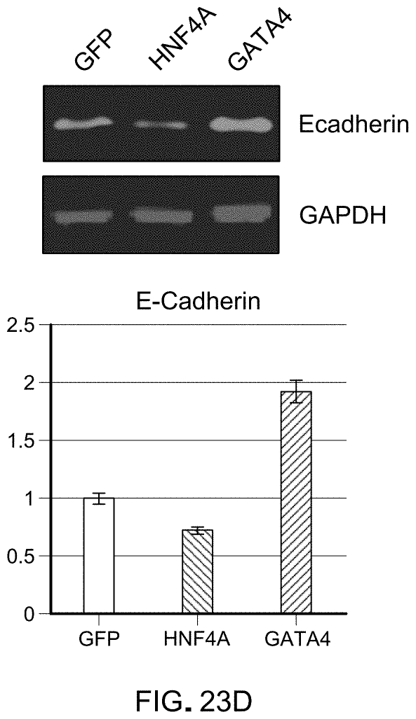

E-cadherin expression was observed in GATA4, Foxa2, HNF4A, and GFP transduced cell lines in vitro and in vivo. GATA4, Foxa2, HNF4A, and GFP transduced cell lines were grown on coverslips and stained with anti-E-cadherin antibody. The immunofluorescence analysis indicated that E-cadherin was expressed more intensely in Foxa2, HNF4A, and GATA4 transduced cells when compared to GFP expressing cells. E-cadherin localized at the cell membrane (FIG. 23A). To examine whether GATA4 expression rescues E-cadherin expression, cell lines that express GATA4, Foxa2, HNF4A, and GFP were harvested, lysed, and analyzed by western blot. E-cadherin was rescued by more than 2-fold in GATA4 expression line (FIG. 23B), reflecting the results obtained by immunofluorescence analysis as well as by western blot (FIG. 23A). An in vivo experiment was carried out to determine the E-cadherin expression level in GATA4, HNF4A, and GFP overexpression tumors. As shown in FIG. 23C and FIG. 23D, tumors with GATA4 overexpression have more than 2-fold higher level of E-cadherin when tested by either immunofluorescence analysis or western blot. E-cadherin expression was increased in the HNF4A overexpressing cell line, but the tumor growth rate was not reduced in comparison with GFP control cell line. These data show that E-cadherin level elevation may be used as an indicator of functional improvement for reprogrammed tumor cells.

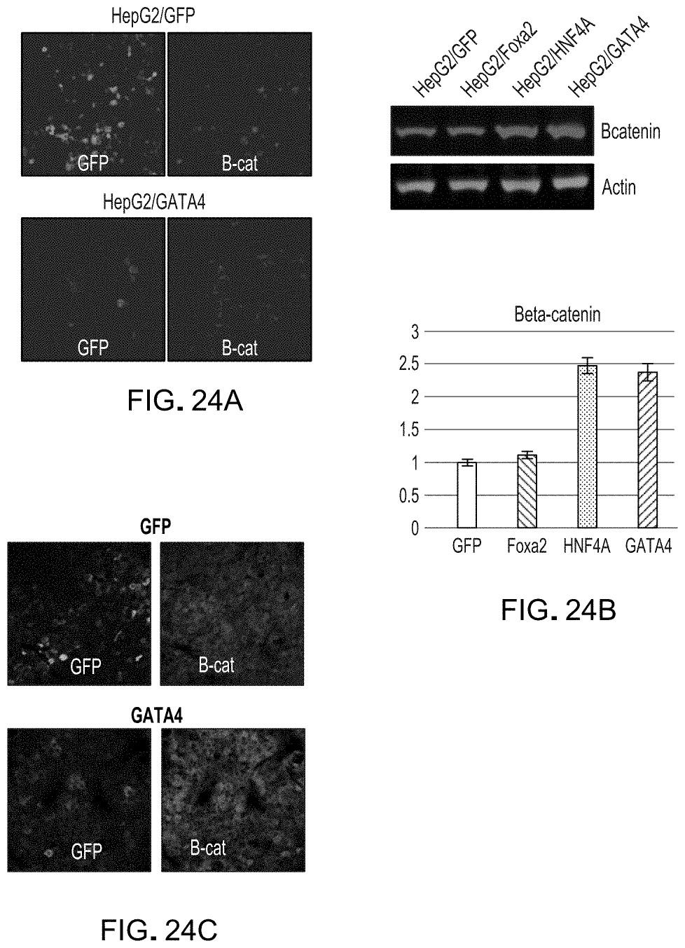

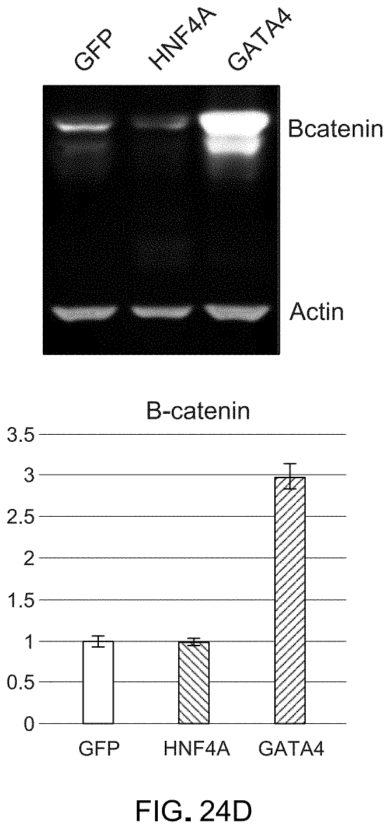

These findings indicate that transcription factor GATA4 promoted E-cadherin expression. To examine whether GATA4 overexpression has effects on beta-catenin, immunofluorescence analysis was performed on both GATA4 and GFP transduced cell lines. GATA4 transduced cell lines had increased beta-catenin expression as compared with GFP overexpressing cells (FIG. 24A). The results also showed that beta-catenin in GFP cells was distributed in perinuclear region, with slight preference for nuclear distribution whereas beta-catenin in GATA4 transduced cell lines was distributed to cell surface. Western blot was used to analyze beta-catenin expression in GATA4 transduced cell lines. GATA4 transduced cell lines had increased beta-catenin expression (FIG. 24B). HNF4A transduced cells also showed increased beta-catenin expression.

Tumor samples from tumors formed in vivo from GATA4 transduced cell lines or GFP transduced cell lines were stained for beta-catenin, and immunofluorescence analysis showed that beta-catenin expression in GATA4 tumors was higher than GFP tumors. Beta-catenin distribution difference between the two kinds of tumors could not be determined due to the crowded and tiny cytoplasm space of the cells in tumors (FIG. 24C). The increased beta-catenin amount in GATA4 tumors was 3-fold higher than GFP tumors, as evaluated by western blot (FIG. 24D).

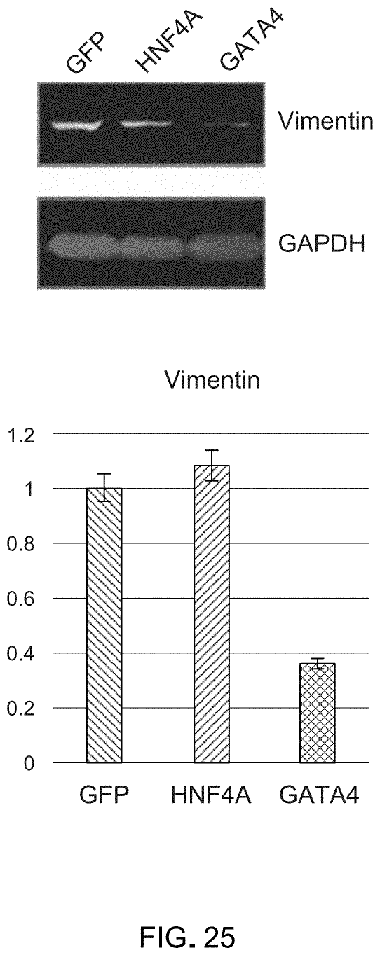

Tumor samples from tumors formed in vivo from GATA4, HNF4A, or GFP transduced cell lines were also analyzed for vimentin by western blot. As shown in FIG. 25, vimentin expression was reduced in GATA4 overexpressing tumors as compared to GFP tumors. Vimentin expression levels in GATA4 tumor were quantified, and the reduction of vimentin in GATA4 tumors was more than 2-fold when compared to GFP tumors.

These findings indicate that HCC tumor cell fate was changed through transcription factor overexpression. The changing mechanism can be associated with mesenchymal to epithelial transformation (MET), a reversed process of epithelial to mesenchymal transition (EMT). Together, these results demonstrate that cancer cells (e.g., liver cancer cells) can be reprogrammed into normal-like cells (e.g., normal-like liver cells) both in vitro and in vivo, leading to an alternative therapeutic approach to treat cancer (e.g., liver cancer).

### Other Aspects

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for treating a mammal having a cancer, wherein said method comprises administering nucleic acid encoding one or more transcription factors to cancer cells within said mammal, wherein said one or more transcription factors are expressed by said cancer cells, and wherein said one or more transcription factors convert said cancer cells into non-cancerous cells within said mammal, thereby reducing the number of cancer cells within said mammal.

2. The method of claim 1, wherein said mammal is a human.

3. The method of claim 1, wherein said cancer is a glioma.

4. The method of claim 3, wherein said one or more transcription factors are one or more neuronal transcription factors.

5. The method of claim 4, said one or more neuronal transcription factors are selected from the group consisting of a neurogenic differentiation factor 1 (NeuroD1) polypeptide, a neurogenin-2 (Neurog2) polypeptide, and an achaete-scute homolog 1 (Ascl1) polypeptide.

6. The method of claim 4, said one or more neuronal transcription factors comprise a NeuroD1 polypeptide, a Neurog2 polypeptide, and an Ascl1 polypeptide.

7. The method of claim 3, wherein said non-cancerous cells are neurons.

8. The method of claim 7, said neurons are FoxG1-positive forebrain neurons.

9. The method of claim 1, wherein said cancer is a liver cancer.

10. The method of claim 9, wherein said liver cancer is a hepatocellular carcinoma.

11. The method of claim 9, wherein said one or more transcription factors are liver transcription factors.

12. The method of claim 10, wherein said one or more liver transcription factors are selected from the group consisting of a hepatocyte nuclear factor 4A (HNF4A) polypeptide, a forkhead box protein (Foxa2) polypeptide, and a GATA binding protein (GATA4) polypeptide.

13. The method of claim 11, wherein said one or more liver transcription factors comprises a HNF4A polypeptide, a Foxa2 polypeptide, and a GATA4 polypeptide.

14. The method of claim 9, wherein said non-cancerous cells are hepatocytes.

15. The method of claim 14, wherein said hepatocytes are hepatocytes that secrete a liver enzyme.

16. The method of claim 15, wherein said liver enzyme is albumin.

17. The method of claim 1, wherein said nucleic acid encoding said one or more transcription factors is administered to said cancer cells in the form of a viral vector.

18. The method of claim 17, wherein said viral vector is a retroviral vector.

19. The method of claim 17, wherein said viral vector is a lentiviral vector.

20. The method of claim 1, wherein said nucleic acid encoding each of said one or more transcription factors is operably linked to a promoter sequence.

21. The method of claim 1, wherein said administration of said nucleic acid encoding said one or more transcription factors comprises a direct injection into a tumor of said mammal.

22. The method of claim 1, wherein said administration of said nucleic acid encoding said one or more transcription factors comprises an intraperitoneal, intramuscular, intravenous, intrathecal, intracerebral, intraparenchymal, intratumoral, intranasal, or oral administration.

23. The method of claim 1, wherein said method comprises, prior to said administering step, identifying said mammal as having said cancer.

24-26. (canceled)
